(19) United States
(12) Patent Application Publication
(10) Pub. No.: US 2023/0338392 A1
(43) Pub. Date: Oct. 26, 2023

(54) SUBCUTANEOUS ADMINISTRATION OF AN ASBT INHIBITOR

(71) Applicant: Albireo AB, Goteborg (SE)

(72) Inventors: Erik Lindström, Mölndal (SE); Jan Mattsson, Kullavik (SE)

(21) Appl. No.: 18/138,375

(22) Filed: Apr. 24, 2023

(30) Foreign Application Priority Data
Apr. 22, 2022 (SE) 2250486-4

(51) Int. Cl. A61K 9/00; A61K 31/554; A61P 1/16

(52) U.S. Cl. CPC A61K 31/554; A61K 9/0019; A61P 1/16

(57) ABSTRACT

The invention relates to an apical sodium-dependent bile acid transporter (ASBT) inhibitor for use in the treatment of a liver or renal disease or disorder, wherein the ASBT inhibitor is administered subcutaneously. Such administration also targets the ASBT in the kidneys and may therefore be useful in the treatment of liver or renal diseases and conditions requiring a stronger inhibition of bile acid circulation, such as in the treatment of cholestatic liver diseases and conditions comprising biliary obstruction or an impaired or defective biliary flow.

### CROSS-REFERENCE TO RELATED APPLICATIONS

This application claims priority to Swedish patent application No. 2250486-4, filed Apr. 22, 2022, the disclosure of which is incorporated herein by reference in its entirety.

### TECHNICAL FIELD

The invention relates to an apical sodium-dependent bile acid transporter (ASBT) inhibitor for use in the treatment of a liver or renal disease or disorder, wherein the ASBT inhibitor is administered subcutaneously. Such administration also targets the ASBT in the kidneys and may therefore be useful in the treatment of liver or renal diseases and conditions requiring a stronger inhibition of bile acid circulation, such as in the treatment of cholestatic liver diseases and conditions comprising biliary obstruction or an impaired or defective biliary flow.

### BACKGROUND

Liver diseases comprise a large number of different diseases, which may be caused by a variety of factors, including viral infections (such as hepatitis); obesity and misuse of alcohol; genetic defects; abnormalities of the immune system (causing e.g. primary biliary cholangitis, primary sclerosing cholangitis and autoimmune hepatitis); and various cancers. If left untreated, liver diseases cause severe damage to the liver (e.g., cirrhosis) and may ultimately result in liver failure.

In several liver diseases, bile acids are involved in disease progression. Bile acids are physiological detergents that play an important role in the intestinal absorption and transport of lipids, nutrients and vitamins. They are also signaling molecules that activate nuclear and membrane-bound receptors and cell signaling pathways that regulate lipid, glucose and energy metabolism. Bile acids are steroid acids that are synthesized from cholesterol in the liver and stored in the gallbladder as mixed micelles. During digestion, the duodenum triggers the release of hormones that cause the gallbladder to contract, thereby releasing bile acids in the small intestine where they enable absorption of fat-soluble vitamins and cholesterol. When they reach the ileum, bile acids are reabsorbed from the intestine and secreted into portal blood to return to the liver via the portal venous circulation. Over 90% of the bile acids are thus recycled and returned to the liver. These bile acids are then transported across the sinusoidal membrane of hepatocytes and re-secreted across the canalicular membrane into bile. In this first pass, 75-90% of bile acids are taken up by hepatocytes, completing one round of enterohepatic circulation. The fraction of bile acids that escapes being cleared in the liver enters the systemic circulation where the free bile acids are filtered by the renal glomerulus, efficiently reclaimed in the proximal tubules and exported back into the systemic circulation. Interestingly, most of the bile acids secreted across the canalicular membrane into bile are derived from the recirculating pool with less than 10% coming from new de novo hepatic synthesis. The small fraction of bile acids that is not reabsorbed in the ileum reaches the colon. Within the intestinal lumen, the primary bile acids (cholic acid and chenodeoxycholic acid) are transformed into secondary bile acids (deoxycholic acid and lithocholic acid) under the action of intestinal bacteria, mainly by single or dual dehydroxylation reactions of the steroid nucleus. The bile acids that escape intestinal absorption are thereafter excreted into the faeces.

The transport of bile acids in the human body (the enterohepatic circulation) is controlled by the action of the members of the SLC10 family of solute carrier proteins, in particular by the Na⁺-taurocholate cotransporting polypeptide (NTCP, also called liver bile acid transporter (LBAT); gene symbol SLC10A1), which is expressed in the sinusoidal membrane of hepatocytes, and by the apical sodium dependent bile acid transporter (ASBT, ISBT, ABAT or NTCP2; gene symbol SLC10A2), which is expressed in the apical membrane of ileal enterocytes, proximal renal tubule cells, biliary epithelium, large cholangiocytes and gallbladder epithelial cells. In the liver, bile acids are efficiently extracted from portal blood by the liver bile acid transporter (LBAT) and re-secreted across the canalicular membrane by the bile salt export pump (BSEP; gene symbol ABCB11). The reabsorption of bile acids in the ileum is handled by the apical sodium-dependent bile acid transporter (ASBT), where it is commonly referred to as ileal bile acid transporter (IBAT). Both LBAT and ASBT function as electrogenic sodium-solute cotransporters that move two or more Na⁺ ions per molecule of solute.

Overall, the efficient transport system helps maintain a constant bile acid pool, ensuring sufficiently high levels of conjugated bile acids in the intestine to promote lipid absorption as well as reduce the small intestinal bacterial load. The system also minimizes fecal and urinary bile acid loss and protects the intestinal and hepatobiliary compartments by eliminating potentially cytotoxic detergents (as reviewed by Kosters and Karpen (Xenobiotica 2008, vol. 38, p. 1043-1071); by Chiang (J. Lipid Res. 2009, vol. 50, p. 1955-1966); and by Dawson (Handb. Exp. Pharmacol. 2011, vol. 201, p. 169-203)).

The regulation of the bile acid pool size has been found to play a key role in cholesterol homeostasis by hepatic conversion of cholesterol to bile acid, which represents a major route for elimination of cholesterol from the body. The liver plays an essential role in removing endogenous and xenobiotic compounds from the body. The normal hepatobiliary secretion and enterohepatic circulation are required for the elimination of endogenous compounds such as cholesterol and bilirubin and their metabolites from the body, thereby maintaining lipid and bile acid homeostasis. (Kosters and Karpen, Xenobiotica 2008, vol. 38, p. 1043-1071).

For a variety of reasons, the bile flow from the liver to the duodenum may be impaired or blocked, thereby causing cholestasis that leads to an accumulation of bile acids in the liver. The cholestasis may originate within the liver (intrahepatic cholestasis) or be caused by blockages in the bile ducts, such as gallstones, cysts, and tumours that restrict the bile flow (extrahepatic or obstructive cholestasis). Cholestasis may lead to drastically elevated serum bile acid levels and may cause jaundice and pruritus.

Numerous compounds capable of inhibiting the reabsorption of bile acids in the ileum (i.e., ASBT or IBAT inhibitors) have been discovered during the past decades; see e.g., WO 97/33882, WO 02/50051, WO 03/022286, WO 2008/058628, WO 2011/137135 and WO 2019/234077. Most of these compounds are not systemically absorbed following oral administration. Whereas ASBT inhibitor compounds initially were contemplated for the treatment of dyslipidemic, metabolic disorder and gastrointestinal diseases (such as constipation; see e.g., WO 2004/089350), it was later discovered that ASBT inhibitors also could be used in the treatment of liver diseases; see e.g. WO 2012/064266, WO 2013/063512 and EP 1535913. Two ASBT inhibitors have recently been approved for treatment of cholestatic liver diseases, namely Bylvay™ (odevixibat) for the treatment of PFIC and pruritus in PFIC, and Livmarli™ (maralixibat) for the treatment of pruritus in Alagille syndrome.

The treatment of additional liver and renal diseases with these and other ASBT inhibitors is currently being developed. Despite recent developments in this field, however, and a slowly increasing number of approved drugs, the treatment of liver diseases remains challenging, especially the more severe forms of cholestatic liver diseases. For these conditions, inhibition of the reabsorption of bile acids in the ileum alone may not be sufficient for providing an effective treatment. There is therefore a continued need for improved treatments of liver diseases.

### BRIEF DESCRIPTION OF THE DRAWINGS

FIG. 1 shows the plasma concentration of elobixibat in mice following subcutaneous (3 or 10 mg/kg) or intravenous (1 mg/kg) administration of elobixibat.

FIG. 2 shows the plasma concentration of elobixibat in mice following subcutaneous administrations of elobixibat (1 mg/kg) at day 1 and day 5.

FIG. 3 is a plot of the total concentration of serum bile acids following treatment of DDC mice with vehicle or different doses of elobixibat for 14 days.

FIG. 4 is a plot of the total concentration of serum bilirubin following treatment of DDC mice with vehicle or different doses of elobixibat for 14 days.

FIG. 5 is a plot of the serum alkaline phosphatase (ALP) levels following treatment of DDC mice with vehicle or different doses of elobixibat for 14 days.

FIG. 6 is a plot of the relative mRNA expression of kidney injury molecule-1 (KIM-1) following treatment of mice with vehicle or different doses of elobixibat for 14 days (left bars - control; right bars - DDC).

FIG. 7 is a plot of the relative mRNA expression of lipocalin-2 (LCN2) following treatment of mice with vehicle or different doses of elobixibat for 14 days (left bars - control; right bars - DDC).

### DETAILED DESCRIPTION OF THE INVENTION

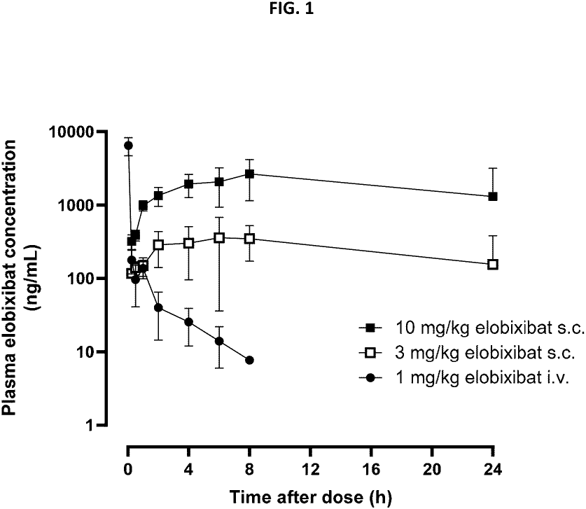

In a first aspect, provided herein is an ASBT inhibitor, or a pharmaceutically acceptable salt thereof, for use in the treatment of a liver or renal disease or disorder, wherein the ASBT inhibitor is administered subcutaneously. It has been found that subcutaneous administration of an ASBT inhibitor, such as elobixibat, results in a high bioavailability of the ASBT inhibitor, with a constant exposure lasting for more than 24 hours. This is surprising, as intravenous administration of the same ASBT inhibitor was found to result in a quick clearance of the compound (see FIG. 1).

As ASBT is predominantly expressed in the ileum (where it is often referred to as IBAT), ASBT inhibitors need not be systemically available for most indications. Indeed, the systemic absorption of the vast majority of known ASBT inhibitors is very low, such as less than 10%. However, since ASBT is also expressed in the proximal tubule cells of the kidneys, systemically available ASBT inhibitors may also inhibit the reuptake of bile acids in the kidneys. It is believed that this may lead to increased levels of bile acids in urine, and to an increased removal of bile acids from the body via the urine. Consequently, systemically available ASBT inhibitors that exert their effect not only in the ileum but also in the kidneys are expected to lead to a greater reduction of bile acid levels than non-systemically available ASBT inhibitors that only exert their effect in the ileum. Under obstructive conditions where bile flow is completely blocked (e.g., due to gallstones, tumours, or inflammation), ASBT inhibitors acting in the ileum are not likely to provide benefit, as there is a very limited amount of bile acids to block in the ileum. By contrast, targeting renal ASBT may be an alternative means of increasing bile acid excretion and reducing hepatic bile acid load. Subcutaneous administration of an ASBT inhibitor may therefore provide a different and possibly longer lasting bile acid modulating effect than oral administration of the ASBT inhibitor under obstructive conditions. Such an effect may be useful in treatment of liver and renal diseases wherein a stronger inhibition of the bile acid circulation is required or when oral administration is not likely to provide benefit (i.e., when bile flow is blocked).

### ASBT Inhibitors

In some embodiments, the ASBT inhibitor for use in the present invention does not inhibit renal ASBT at clinically relevant levels following oral administration of such ASBT inhibitor.

In some embodiments, the systemic absorption of such ASBT inhibitor following oral administration is less than 10%, such as less than 9%, such as less than 8%, such as less than 7%, or such as less than 6%. In some embodiments, the systemic absorption of such ASBT inhibitor following oral administration is less than 5%. In some embodiments, the systemic absorption of such ASBT inhibitor following oral administration is less than 1%.

In some embodiments, the ASBT inhibitor is a compound disclosed in, e.g., WO 93/16055, WO 94/18183, WO 94/18184, WO 96/05188, WO 96/08484, WO 96/16051, WO 97/33882, WO 98/03818, WO 98/07449, WO 98/40375, WO 99/35135, WO 99/64409, WO 99/64410, WO 00/01687, WO 00/47568, WO 00/61568, WO 00/38725, WO 00/38726, WO 00/38727, WO 00/38728, WO 00/38729, WO 01/66533, WO 01/68096, WO 02/32428, WO 02/50051, WO 03/020710, WO 03/022286, WO 03/022825, WO 03/022830, WO 03/061663, WO 03/091232, WO 03/106482, WO 2004/006899, WO 2004/076430, WO 2007/009655, WO 2007/009656, WO 2008/058628, WO 2008/058630, WO 2011/137135, WO 2019/234077, WO 2020/161216, WO 2020/161217, WO 2021/110884, WO 2021/110885, WO 2021/110886, WO 2021/110887, WO 2022/029101, DE 19825804, EP 864582, EP 489423, EP 549967, EP 573848, EP 624593, EP 624594, EP 624595, EP 624596, EP 0864582, EP 1173205, EP 1535913, EP 1719768 or EP 3210977.

In some embodiments, the ASBT inhibitor is a compound of formula (I):

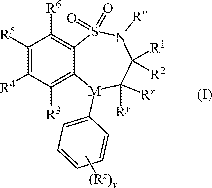

wherein:
R^{v} is selected from hydrogen or C₁₋₆alkyl;
One of R¹ and R² is selected from hydrogen, C₁₋₆alkyl or C₂₋₆alkenyl and the other is selected from C₁₋₆alkyl or C₂₋₆alkenyl;
R^{x} and R^{y} are independently selected from the group consisting of hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2;
M is selected from -N- or -CH-;
R^{z} is selected from the group consisting of halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;
v is 0-5;
one of R⁴ and R⁵ is a group of formula (IA):

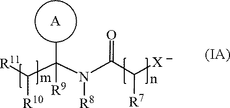

R³ and R⁶ and the other of R⁴ and R⁵ are independently selected from the group consisting of hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R³ may be optionally substituted on carbon by one or more R¹⁶;
X is -O-, -N(R^{a})-, -S(O)_{b}- or -CH(R^{a})-; wherein R^{a} is hydrogen or C₁₋₆alkyl and b is 0-2;
Ring A is aryl or heteroaryl; wherein Ring A is optionally substituted by one or more substituents selected from R¹⁷;
R⁷ is hydrogen, C₁₋₄alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R¹⁰ is hydrogen, C₁₋₄alkyl, carbocyclyl or heterocyclyl; wherein R¹⁰ is optionally substituted by one or more substituents selected from R¹⁹;
R¹¹ is carboxy, sulpho, sulphino, phosphono, -P(O)(OR^{c})(OR^{d}), -P(O)(OH)(OR^{c}), -P(O)(OH)(R^{d}) or -P(O)(OR^{c})(R^{d}) wherein R^{c} and R^{d} are independently selected from C₁₋₆alkyl; or R¹¹ is a group of formula (IB) or (IC):

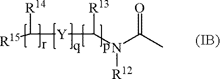

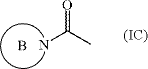

wherein:
   Y is -N(Rⁿ)-, -N(Rⁿ)C(O)-, -N(Rⁿ)C(O)(CR^{s}R^{t})ᵥN(Rⁿ)C(O)-, -O-, and -S(O)a-; wherein a is 0-2, v is 1-2, R^{s} and R^{t} are independently selected from hydrogen or C₁₋₄alkyl optionally substituted by R²⁶ and Rⁿ is hydrogen or C₁₋₄alkyl;
   R¹² is hydrogen or C₁₋₄alkyl;
   R¹³ and R¹⁴ are independently selected from hydrogen, C₁₋₆alkyl, carbocyclyl or heterocyclyl; and when q is 0, R¹⁴ may additionally be selected from hydroxy; wherein R¹³ and R¹⁴ may be independently optionally substituted by one or more substituents selected from R²⁰;
   R¹⁵ is carboxy, sulpho, sulphino, phosphono, -P(O)(OR^{e})(OR^{f}), -P(O)(OH)(OR^{e}), -P(O)(OH)(R^{e}) or —P(O)(OR^{e})(R^{f}) wherein R^{e} and R^{f} are independently selected from C₁₋₆alkyl;
   p is 1-3; wherein the values of R¹³ may be the same or different;
   q is 0-1;
   r is 0-3; wherein the values of R¹⁴ may be the same or different;
   m is 0-2; wherein the values of R¹⁰ may be the same or different;
   n is 1-3; wherein the values of R⁷ may be the same or different;
   Ring B is a nitrogen linked heterocyclyl substituted on carbon by one group selected from R²³, and optionally additionally substituted on carbon by one or more R²⁴; and wherein if said nitrogen linked heterocyclyl contains an -NH- moiety, that nitrogen may be optionally substituted by a group selected from R²⁵;
   R¹⁶, R¹⁷ and R¹⁸ are independently selected from the group consisting of halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;
   R¹⁹, R²⁰, R²⁴ and R²⁶ are independently selected from the group consisting of halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, benzyloxycarbonylamino, (C₁₋₄alkyl)₃silyl, sulpho, sulphino, amidino, phosphono, -P(O)(OR^{a})(OR^{b}), -P(O)(OH)(OR^{a}), -P(O)(OH)(R^{a}) or -P(O)(OR^{a})(R^{b}), wherein R^{a} and R^{b} are independently selected from C₁₋₆alkyl; wherein R¹⁹, R²⁰, R²⁴ and R²⁶ may be independently optionally substituted on carbon by one or more R²²;
   R²¹ and R²² are independently selected from the group consisting of halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
   R²³ is carboxy, sulpho, sulphino, phosphono, -P(O)(OR^{g})(OR^{h}), -P(O)(OH)(OR^{g}), -P(O)(OH)(R^{g}) or -P(O)(OR^{g})(R^{h}) wherein R^{g} and R^{h} are independently selected from C₁₋₆alkyl; and
   R²⁵ is selected from the group consisting of C₁₋₆alkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;
or a pharmaceutically acceptable salt thereof.

In some embodiments, the ASBT inhibitor is a compound of formula (II):

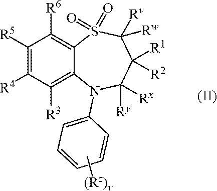

wherein:
R^{v} and R^{w} are independently selected from hydrogen or C₁₋₆alkyl;
R¹ and R² are independently selected from C₁₋₆alkyl;
R^{x} and R^{y} are independently selected from hydrogen or C₁₋₆alkyl, or one of R^{x} and R^{y} is hydrogen or C₁₋₆alkyl and the other is hydroxy or C₁₋₆alkoxy;
R^{z} is selected from the group consisting of halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl and N,N-(C₁₋₆alkyl)₂sulphamoyl;
v is 0-5;
one of R⁴ and R⁵ is a group of formula (IIA):

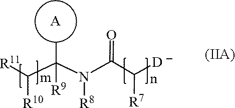

R³ and R⁶ and the other of R⁴ and R⁵ are independently selected from the group consisting of hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂-amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl;
wherein R³ and R⁶ and the other of R⁴ and R⁵ may be optionally substituted on carbon by one or more R¹⁶;
D is -O-, -N(R^{a})-, -S(O)_{b}- or -CH(R^{a})-; wherein R^{a} is hydrogen or C₁₋₆alkyl and b is 0-2;
Ring A is aryl or heteroaryl; wherein Ring A is optionally substituted by one or more substituents selected from R¹⁷;
R⁷ is hydrogen, C₁₋₄alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R¹⁰ is hydrogen,C₁₋₄alkyl, carbocyclyl or heterocyclyl; wherein R¹⁰ is optionally substituted by one or more substituents selected from R¹⁹;
R¹¹ is selected from the group consisting of carboxy, sulpho, sulphino, phosphono, tetrazolyl, -P(O)(OR^{c})(OR^{d}), -P(O)(OH)(OR^{c}), -P(O)(OH)(R^{d}) and -P(O)(OR^{c})(R^{d}) wherein R^{c} and R^{d} are independently selected from C₁₋₆alkyl; or R¹¹ is a group of formula (IIB):

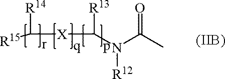

wherein:
   X is -N(R^{q})-, -N(R^{q})C(O)-, -O-, or -S(O)ₐ-; wherein a is 0-2 and R^{q} is hydrogen or C₁₋₄alkyl;
   R¹² is hydrogen or C₁₋₄alkyl;
   R¹³ and R¹⁴ are independently selected from the group consisting of hydrogen, C₁₋₄alkyl, carbocyclyl, heterocyclyl and R²³; wherein said C₁₋₄alkyl, carbocyclyl or heterocyclyl may be independently optionally substituted by one or more substituents selected from R²⁰;
   R¹⁵ is selected from the group consisting of carboxy, sulpho, sulphino, phosphono, tetrazolyl, -P(O)(OR^{e})(OR^{f}), -P(O)(OH)(OR^{e}), -P(O)(OH)(R^{e}) and -P(O)(OR^{e})(R^{f}) wherein R^{e} and R^{f} are independently selected from C₁₋₆alkyl; or R¹⁵ is a group of formula (IIC): 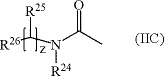
   wherein:
      R²⁴ is hydrogen or C₁₋₄alkyl;
      R²⁵ is selected from the group consisting of hydrogen, C₁₋₄alkyl, carbocyclyl, heterocyclyl and R²⁷; wherein said C₁₋₄alkyl, carbocyclyl or heterocyclyl may be independently optionally substituted by one or more substituents selected from R²⁸;
      R²⁶ is selected from the group consisting of carboxy, sulpho, sulphino, phosphono, tetrazolyl, -P(O)(OR^{g})(OR^{h}), -P(O)(OH)(OR^{g}), -P(O)(OH)(R^{g}) and -P(O)(OR^{g})(R^{h}) wherein R^{g} and R^{h} are independently selected from C₁₋₆alkyl;
      p is 1-3; wherein the values of R¹³ may be the same or different;
      q is 0-1;
      r is 0-3; wherein the values of R¹⁴ may be the same or different;
      m is 0-2; wherein the values of R¹⁰ may be the same or different;
      n is 1-3; wherein the values of R⁷ may be the same or different;
      z is 0-3; wherein the values of R²⁵ may be the same or different;
      R¹⁶, R¹⁷ and R¹⁸ are each independently selected from the group consisting of halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl;
      wherein R¹⁶, R¹⁷ and R¹⁸ may be independently optionally substituted on carbon by one or more R²¹;
      R¹⁹, R²⁰, R²³, R²⁷ and R²⁸ are each independently selected from the group consisting of halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-C₁₋₄alkyl)sulphamoyl, N,N-(C₁₋₄alkyl)₂sulphamoyl, carbocyclyl, heterocyclyl, sulpho, sulphino, amidino, phosphono, -P(O)(OR^{a})(OR^{b}), -P(O)(OH)(OR^{a}), -P(O)(OH)(R^{a}) or -P(O)(OR^{a})(R^{b}), wherein R^{a} and R^{b} are independently selected from C₁₋₆alkyl; wherein R¹⁹, R²⁰, R²³, R²⁷ and R²⁸ may be independently optionally substituted on carbon by one or more R²²;
      R²¹ and R²² are independently selected from the group consisting of halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
or a pharmaceutically acceptable salt thereof.

In some embodiments, the ASBT inhibitor is a compound of formula (III):

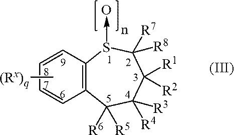

wherein:
q is an integer from 1 to 4;
n is an integer from 0 to 2;
R ¹ and R² are independently selected from the group consisting of H, alkyl, alkenyl, alkynyl, haloalkyl, alkylaryl, arylalkyl, alkoxy, alkoxyalkyl, dialkylamino, alkylthio, (polyalkyl)aryl, and cycloalkyl,
wherein alkyl, alkenyl, alkynyl, haloalkyl, alkylaryl, arylalkyl, alkoxy, alkoxyalkyl, dialkylamino, alkylthio, (polyalkyl)aryl, and cycloalkyl optionally are substituted with one or more substituents selected from the group consisting of OR⁹, NR⁹R¹⁰, N⁺R⁹R¹⁰R^{w}A⁻, SR⁹, S⁺R⁹R¹⁰A⁻. P⁺R⁹R¹⁰R¹¹A⁻, S(O)R⁹, SO₂R⁹, SO₃R⁹, CO₂R⁹, CN, halogen, oxo, and CONR⁹R¹⁰,
wherein alkyl, alkenyl, alkynyl, alkylaryl, alkoxy, alkoxyalkyl, (polyalkyl)aryl, and cycloalkyl optionally have one or more carbons replaced by O, NR⁹, N⁺R⁹R¹⁰A⁻, S, SO, SO₂, S⁺R⁹A⁻, P⁺R⁹R¹⁰A⁻, or phenylene,
wherein R⁹, R¹⁰, and R^{w} are independently selected from the group consisting of H, alkyl, alkenyl, alkynyl, cycloalkyl, aryl, acyl, heterocycle, ammoniumalkyl, arylalkyl, carboxyalkyl, carboxyheteroaryl, carboxyheterocycle, carboalkoxyalkyl, carboxyalkylamino, heteroarylalkyl, heterocyclylalkyl, and alkylammoniumalkyl; or
R¹ and R² taken together with the carbon to which they are attached form C₃₋₁₀cycloalkyl;
R³ and R⁴ are independently selected from the group consisting of H, alkyl, alkenyl, alkynyl, acyloxy, aryl, heterocycle, OR⁹, NR⁹R¹⁰, SR⁹, S(O)R⁹, SO₂R⁹, and SO₃R⁹, wherein R⁹ and R¹⁰ are as defined above; or
R³ and R⁴ together form =O, =NOR¹¹, =S, =NNR¹¹R¹², =NR⁹, or =CR¹¹R¹²,
wherein R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R⁵ is aryl substituted with one or more OR^{13a},
wherein R^{13a} is selected from the group consisting of alkylarylalkyl, alkylheteroarylalkyl, alkylheterocyclylalkyl, heterocyclylalkyl, heteroarylalkyl, quaternary heterocyclylalkyl, alkylammoniumalkyl, and carboxyalkylaminocarbonylalkyl,
R^{13a} is optionally substituted with one or more groups selected from the group consisting of hydroxy, amino, sulfo, carboxy, alkyl, carboxyalkyl, heterocycle, heteroaryl, sulfoalkyl, quaternary heterocycle, quaternary heteroaryl, quaternary heterocyclylalkyl, quaternary heteroarylalkyl, guanidinyl, OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹⁰R¹¹A⁻, S⁺R⁹R¹⁰A⁻, and C(O)OM,
wherein A⁻ is a pharmaceutically acceptable anion and M is a pharmaceutically acceptable cation, wherein R¹⁶ and R¹⁷ are independently selected from the substituents constituting R⁹ and M; and
R⁶ is selected from the group consisting of H, alkyl, alkenyl, alkynyl, aryl, cycloalkyl, heterocycle, quaternary heterocycle, OR³⁰, SR⁹, S(O)R⁹, SO₂R⁹, and SO₃R⁹,
wherein alkyl, alkenyl, alkynyl, aryl, cycloalkyl, heterocycle, quaternary heterocycle, and quaternary heteroaryl can be substituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, COR¹³, NR¹³C(O)R¹⁴, NR¹³C(O)NR¹⁴R¹⁵, NR¹³CO₂R¹⁴, OC(O)R¹³, OC(O)NR¹³R¹⁴, NR¹³SOR¹⁴, NR¹³SO₂R¹⁴, NR¹³SONR¹⁴R¹⁵, NR¹³SO₂NR¹⁴R¹⁵, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻, wherein:
A⁻ is a pharmaceutically acceptable anion and M is a pharmaceutically acceptable cation,
said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, SR⁷, S(O)R⁷, SO₂R⁷, SO₃R⁷, CO₂R⁷, CN, oxo, CONR⁷R⁸, N⁺R⁷R³R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(0)(OR⁷)OR⁸, and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A, or phenylene, and R¹³, R¹⁴, and R¹⁵ are independently selected from the group consisting of hydrogen, alkyl, alkenyl, alkynyl, polyalkyl, polyether, aryl, arylalkyl, alkylarylalkyl, alkylheteroarylalkyl, alkylheterocyclylalkyl, cycloalkyl, heterocycle, heteroaryl, quaternary heterocycle, quaternary heteroaryl, heterocyclylalkyl, heteroarylalkyl, quaternary heterocyclylalkyl, quaternary heteroarylalkyl, alkylammoniumalkyl, and carboxyalkylaminocarbonylalkyl,
wherein alkyl, alkenyl, alkynyl, arylalkyl, heterocycle, and polyalkyl optionally have one or more carbons replaced by O, NR⁹, N⁺R⁹R¹⁰A⁻, S, SO, SO₂, S⁺R⁹A⁻, PR⁹, P⁺R⁹R¹⁰A⁻, P(O)R⁹, phenylene, carbohydrate, amino acid, peptide, or polypeptide, and
R¹³, R¹⁴, and R¹⁵ are optionally substituted with one or more groups selected from the group consisting of hydroxy, amino, sulfo, carboxy, alkyl, carboxyalkyl, heterocycle, heteroaryl, sulfoalkyl, quaternary heterocycle, quaternary heteroaryl, quaternary heterocyclylalkyl, quaternary heteroarylalkyl, guanidinyl, OR⁹, NR⁹R¹⁰, N⁺R⁹R¹¹R¹²A, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹⁰R¹¹A⁻, S⁺R⁹R¹⁰A⁻, and C(O)OM,
wherein R¹⁶ and R¹⁷ are independently selected from the substituents constituting R⁹ and M; or
R¹³ and R¹⁴, together with the nitrogen atom to which they are attached form a mono- or polycyclic heterocycle that is optionally substituted with one or more radicals selected from the group consisting of oxo, carboxy and quaternary salts; or
R¹⁴ and R¹⁵, together with the nitrogen atom to which they are attached, form a cyclic ring; and
R³⁰ is selected from the group consisting of alkyl, alkenyl, alkynyl, cycloalkyl, aryl, acyl, heterocycle, ammoniumalkyl, alkylammoniumalkyl, arylalkyl, carboxyalkyl, carboxyheteroaryl, carboxyheterocycle, carboalkoxyalkyl, carboxyalkylamino, heteroarylalkyl, heterocyclylalkyl, and alkylammoniumalkyl; and
R⁷ and R⁸ are independently selected from the group consisting of hydrogen and alkyl; and
one or more R^{x} are independently selected from the group consisting of H, alkyl, alkenyl, alkynyl, polyalkyl, acyloxy, aryl, arylalkyl, halogen, haloalkyl, cycloalkyl, heterocycle, heteroaryl, polyether, quaternary heterocycle, quaternary heteroaryl, OR¹³, NR¹³R¹⁴, SR¹³, S(O)R¹³, S(O)₂R¹³, SO₃R¹³, S⁺R¹³R¹⁴A⁻, NR¹³OR¹⁴, NR¹³NR¹⁴R¹⁵, NO₂, CO₂R¹³, CN, OM, SO₂OM, SO₂NR¹³R¹⁴, NR¹⁴C(O)R¹³, C(O)NR¹³R¹⁴, NR¹⁴C(O)R¹³, C(O)OM, COR¹³, OR¹⁸, S(O)ₙNR¹⁸, NR¹³R¹⁸, NR¹⁸OR¹⁴, N⁺R⁹R¹¹R¹²A⁻, P⁺R⁹R¹¹R¹²A⁻, amino acid, peptide, polypeptide, and carbohydrate,
wherein alkyl, alkenyl, alkynyl, cycloalkyl, aryl, polyalkyl, heterocycle, acyloxy, arylalkyl, haloalkyl, polyether, quaternary heterocycle, and quaternary heteroaryl can be further substituted with OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹¹R¹²A⁻, S⁺R⁹R¹⁰A⁻, or C(O)OM, and
wherein R¹⁸ is selected from the group consisting of acyl, arylalkoxycarbonyl, arylalkyl, heterocycle, heteroaryl, alkyl,
wherein acyl, arylalkoxycarbonyl, arylalkyl, heterocycle, heteroaryl, alkyl, quaternary heterocycle, and quaternary heteroaryl optionally are substituted with one or more substituents selected from the group consisting of OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₃R⁹, SO₂OM, SO₂NR⁹R¹⁰, PO(OR¹⁶)OR¹⁷, and C(O)OM,
wherein in R^{x}, one or more carbons are optionally replaced by O, NR¹³, N⁺R¹³R¹⁴A⁻, S, SO, SO₂, S⁺R¹³A⁻, PR¹³, P(O)R¹³, P⁺R¹³R¹⁴A⁻, phenylene, amino acid, peptide, polypeptide, carbohydrate, polyether, or polyalkyl,
wherein in said polyalkyl, phenylene, amino acid, peptide, polypeptide, and carbohydrate, one or more carbons are optionally replaced by O, NR⁹, N⁺R⁹R¹⁰A⁻, S, SO, SO₂, S⁺R⁹A⁻, PR⁹, P⁺R⁹R¹⁰A⁻, or P(O)R⁹;
wherein quaternary heterocycle and quaternary heteroaryl are optionally substituted with one or more groups selected from the group consisting of alkyl, alkenyl, alkynyl, polyalkyl, polyether, aryl, haloalkyl, cycloalkyl, heterocycle, arylalkyl, halogen, oxo, OR¹³, NR¹³R¹⁴, SR¹³, S(O)R¹³, SO₂R¹³, SO₃R¹³, NR¹³OR¹⁴, NR¹³NR¹⁴R¹³, NO₂, CO₂R¹³, CN, OM, SO₂OM, SO₂NR¹³R¹⁴, C(O)NR¹³R¹⁴, C(O)OM, COR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻;
or a pharmaceutically acceptable salt thereof.

In some embodiments, the ASBT inhibitor is a compound of formula (IV):

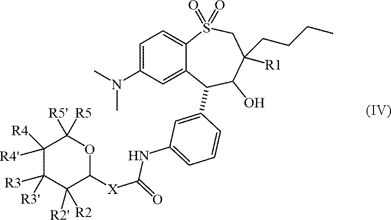

wherein
X is O, NH, CH₂ or a bond;
R¹ is C₁₋₆alkyl;
R², R^{2′}, R³, R^{3′}, R⁴, R^{4′}, R⁵ and R^{5′} are each independently selected from the group consisting of H, Cl, Br, I, OH, -(CH₂)-OH, CF₃, NO₂, N₃, CN, S(O)ₚ-R⁶, O-S(O)ₚ-R⁶, C₁₋₆alkylene-S(O)ₚ-R⁶, C₁₋₆alkylene-O-S(O)ₚ-R⁶, COOH, COOC₁₋₆alkyl, CONH₂, CONHC₁₋₆alkyl, CON(C₁₋₆alkyl)₂, C₁₋₆alkyl, C₂₋₆alkenyl, C₂₋₆alkynyl and O-C₁₋₆alkyl, wherein one or more of the alkyl hydrogens may be replaced by fluorine; and phenyl, -(CH₂)-phenyl, -(CH₂)ₙ-phenyl, O-phenyl, O-(CH₂)ₘ-phenyl, -(CH₂)-O-(CH₂)ₘ-phenyl, wherein the phenyl ring may be substituted one to three times by F, Cl, Br, I, OH, CF₃, NO₂, CN, OCF₃, O-C₁₋₆alkyl, C₁₋₆alkyl, NH₂, NHC₁₋₆alkyl, N(C₁₋₆alkyl)₂, SO₂-CH₃, COOH, COOC₁₋₆alkyl, or CONH₂;
wherein always at least one of R², R^{2′}, R³, R^{3′}, R⁴, R^{4′}, R⁵, R^{5′} is -O-(CH₂)ₘ-phenyl or -(CH₂)-O-(CH₂)ₘ-phenyl, wherein the phenyl ring may be substituted one to 3 times by F, Cl, Br, I, OH, CF₃, NO₂, CN, OCF₃, O-C₁₋₆alkyl, C₁₋₆alkyl, NH₂, NHC₁₋₆alkyl, N(C₁₋₆alkyl)₂, SO₂-CH₃, COOH, COOC₁₋₆alkyl, CONH₂;
R⁶ is selected from the group consisting of H, OH, C₁₋₆alkyl, NH₂, NHC₁₋₆alkyl and N(C₁₋₆alkyl)₂;
n is an integer 2, 3, 4, 5 or 6;
m is an integer 1, 2, 3, 4, 5 or 6; and
p is an integer 0, 1 or 2;
or a pharmaceutically acceptable salt thereof.

In some embodiments, the ASBT inhibitor is a compound of formula (V):

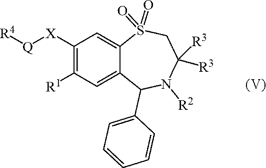

wherein
R¹ is selected from the group consisting of H, Cl, Br, N(CH₃)₂ and methoxy;
R² is H or OH;
each R³ is independently C₁₋₆alkyl;
X is CH₂, C(O) or CH=CH;
Q is C₀₋₆alkyl;
R⁴ is selected from the group consisting of OH, SO₃H, CO₂H, PO₃H₂, CONR⁵R⁵, NR³R^{S} and NHC(O)CH²NR⁵R⁵;
each R⁵ is independently selected from the group consisting of H, OH, C₁₋₆alkyl, C₀₋₆alkylCO₂H, C₀₋₆alkylSO₃H, C₀₋₆alkylPO₃H₂, C(O)C₀₋₆alkylCO₂H, C(O)C₀₋₆alkylSO₃H, C(O)C₀₋₆alkylPO₃H₂ and CH(R⁶)C₀₋₆alkylCO₂H; and
R⁶ is selected from the group consisting of C₀₋₆alkylCO₂H, C₀₋₆alkylOH, C₀₋₆alkylSO₃H and C₀₋₆alkylPO₃H₂;
or a pharmaceutically acceptable salt thereof.

In some embodiments, the ASBT inhibitor is a compound of formula (VI):

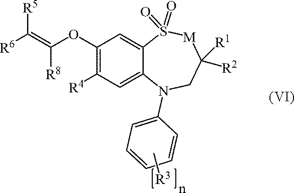

wherein
M is selected from -CH₂- and -NR⁷-;
R¹ and R² are each independently C₁₋₄alkyl;
R³ is selected from the group consisting of hydrogen, halogen, hydroxy, C₁₋₄alkyl, C₁₋₄haloalkyl, C₁₋₄alkoxy, cyano, nitro, amino, N-(C₁₋₄alkyl)amino, N,N-di(C₁₋₄alkyl)amino, N-(aryl-C₁₋₄alkyl)amino, C₁₋₆alkylcarbonylamino, C₃₋₆cycloalkylcarbonylamino, N-(C₁₋₄alkyl)aminocarbonyl, N,N-di(C₁₋₄alkyl)aminocarbonyl, C₁₋₄alkyloxycarbonylamino, C₃₋₆cycloalkyloxycarbonylamino, C₁₋₄alkylsulfonamido and C₃₋₆cycloalkylsulfonamido;
n is an integer 1, 2 or 3;
R⁴ is selected from the group consisting of hydrogen, halogen, cyano, C₁₋₄alkyl, C₃₋₆cycloalkyl, C₁₋₄alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino;
One of R⁵ and R⁶ is carboxy, and the other of R⁵ and R⁶ is selected from the group consisting of hydrogen, fluoro, C₁₋₄alkyl and C₁₋₄haloalkyl; and
R⁷ is selected from the group consisting of hydrogen and C₁₋₄alkyl;
R⁸ is selected from the group consisting of hydrogen and C₁₋₄alkyl;
or a pharmaceutically acceptable salt thereof.

In some embodiments, the ASBT inhibitor is a compound of formula (VII):

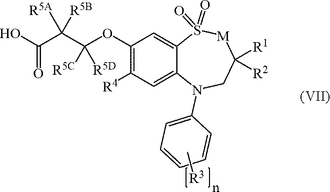

wherein
M is -CH₂- or -NR⁶-;
R¹ and R² are each independently C₁₋₄alkyl;
R³ is independently selected from the group consisting of hydrogen, halogen, hydroxy, C₁₋₄alkyl, C₁₋₄haloalkyl, C₁₋₄alkoxy, C₁₋₄haloalkoxy, cyano, nitro, amino, N-(C₁₋₄alkyl)amino, N,N-di(C₁₋₄alkyl)amino and N-(aryl-C₁₋₄alkyl)amino;
n is an integer 1, 2 or 3;
R⁴ is selected from the group consisting of hydrogen, halogen, hydroxy, cyano, C₁₋₄alkyl, C₃₋₆cycloalkyl, C₁₋₄alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino; and
R^{5A}, R^{5B}, R^{5C} and R^{5D} are each independently selected from the group consisting of hydrogen, halogen, hydroxy, amino, C₁₋₄alkyl and C₁₋₄alkoxy; and
R⁶ is selected from the group consisting of hydrogen and C₁₋₄alkyl;
or a pharmaceutically acceptable salt thereof;

In some embodiments, the ASBT inhibitor is a compound of formula (VIII):

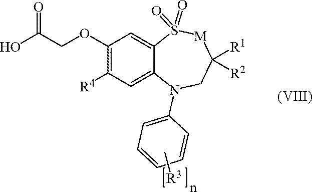

wherein
M is -CH₂- or -NH-;
R¹ and R² are each independently C₁₋₄alkyl;
R³ is independently selected from the group consisting of hydrogen, halogen, hydroxy, C₁₋₄alkyl, C₁₋₄haloalkyl, C₁₋₄alkoxy, C₁₋₄haloalkoxy, cyano, nitro, amino, N-(C₁₋₄alkyl)amino, N,N-di(C₁₋₄alkyl)amino, and N-(aryl-C₁₋₄alkyl)amino;
n is an integer 1, 2 or 3;
R⁴ is selected from the group consisting of hydrogen, halogen, hydroxy, cyano, C₁₋₄alkyl, C₃₋₆cycloalkyl, C₁₋₄alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino;
or a pharmaceutically acceptable salt thereof.

In some embodiments, the ASBT inhibitor is a compound of formula (IX):

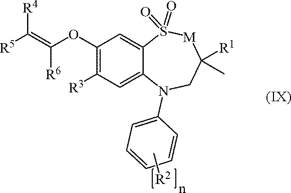

wherein
M is selected from -CH₂- and -NR⁶-;
R¹ is C₁₋₄alkyl;
R² is independently selected from the group consisting of hydrogen, halogen, hydroxy, C₁₋₄alkyl, C₁₋₄haloalkyl, C₁₋₄alkoxy, cyano, nitro, amino, N-(C₁₋₄alkyl)amino, N,N-di(C₁₋₄alkyl)amino, N-(aryl-C₁₋₄alkyl)amino, C₁₋₆alkylcarbonylamino, C₃₋₆cycloalkylcarbonylamino, N-(C₁₋₄alkyl)aminocarbonyl, N,N-di(C₁₋₄alkyl)aminocarbonyl, C₁₋₄alkyloxycarbonylamino, C₃₋₆cycloalkyloxycarbonylamino, C₁₋₄alkylsulfonamido and C₃₋₆cycloalkylsulfonamido;
n is an integer 1, 2 or 3;
R³ is selected from the group consisting of hydrogen, halogen, cyano, C₁₋₄alkyl, C₃₋₆cycloalkyl, C₁₋₄ alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino;
One of R⁴ and R⁵ is carboxyl, and the other of R⁴ and R⁵ is selected from the group consisting of hydrogen, fluoro, C₁₋₄alkyl and C₁₋₄haloalkyl;
R⁶ is selected from the group consisting of hydrogen and C₁₋₄alkyl; and
R⁷ is selected from the group consisting of hydrogen and C₁₋₄alkyl;
or a pharmaceutically acceptable salt thereof.

In some embodiments, the ASBT inhibitor is a compound selected from the group consisting of:
1,1-dioxo-3,3-dibutyl-5-phenyl-7-methylthio-8-(N-{(R)-1′-phenyl-1′-[N′-(carboxymethyl)-carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N-((S)-1-carboxypropyl)carbamoyl]-4-hydroxybenzyl}carbamoylmethoxy)-2,3,4,5-tetrahydro-1,2,5-benzothiadiazepine;
1-{[4-({4-[(4R,5R)-3,3-dibutyl-7-(dimethylamino)-4-hydroxy-1,1-dioxo-2,3,4,5-tetrahydro-1H-1λ⁶-benzothiepin-5-yl]phenoxy}methyl)phenyl]methyl}-1,4-diazabicyclo[2.2.2]octan-1-ium chloride;
N-(3-O-benzyl-6-O-sulfo-β-D-glucopyranosyl)-N′-{3-[(3S,4R,5R)-3-butyl-7-(dimethylamino)-3-ethyl-4-hydroxy-1,1-dioxo-2,3,4,5-tetrahydro-1H-1λ⁶ -benzothiepin-5-yl]phenyl}urea;
3-({[(3R,5R)-3-butyl-3-ethyl-7-methoxy-1,1-dioxo-5-phenyl-2,3,4,5-tetrahydro-1H-1λ⁶,4-benzothiazepin-8-yl]methyl}amino)pentanedioic acid;
(Z)-3-((3,3-dibutyl-7-(methylthio)-1,1-dioxido-5-phenyl-2,3,4,5-tetrahydro-1,5-benzothiazepin-8-yl)oxy)-2-fluoroacrylic acid;
(Z)-3-((3-butyl-3-ethyl-7-(methylthio)-1,1-dioxido-5-phenyl-2,3,4,5-tetrahydro-1,5-benzothiazepin-8-yl)oxy)-2-fluoroacrylic acid;
3-((7-Bromo-3-butyl-3-ethyl-1,1-dioxido-5-phenyl-2,3,4,5-tetrahydro-1,5-benzothiazepin-8-yl)oxy)propanoic acid;
3-((3,3-Dibutyl-7-(methylthio)-1,1-dioxido-5-phenyl-2,3,4,5-tetrahydro-1,5-benzothiazepin-8-yl)oxy)-2-hydroxypropanoic acid;
3-((3-Butyl-3-ethyl-7-(methylthio)-1,1-dioxido-5-phenyl-2,3,4,5-tetrahydro-1,5-benzothiazepin-8-yl)oxy)propanoic acid;
3-((3,3-Dibutyl-7-(methylthio)-1,1-dioxido-5-phenyl-2,3,4,5-tetrahydro-1,2,5-benzothiadiazepin-8-yl)oxy)propanoic acid;
3-((3-butyl-3-ethyl-7-(methylthio)-1,1-dioxido-5-phenyl-2,3,4,5-tetrahydro-1,2,5-benzothiadiazepin-8-yl)oxy)propanoic acid;
2-((3-butyl-7-(dimethylamino)-3-ethyl-1,1-dioxido-5-phenyl-2,3,4,5-tetrahydro-1,5-benzothiazepin-8-yl)oxy)acetic acid;
2-((3-butyl-3-ethyl-7-(methylthio)-1,1-dioxido-5-phenyl-2,3,4,5-tetrahydrobenzo-1,2,5-thiadiazepin-8-yl)oxy)acetic acid; and
(E)-3-((3-butyl-3-ethyl-5-(4-fluorophenyl)-7-(methylthio)-1,1-dioxido-2,3,4,5-tetrahydro-1,5-benzothiazepin-8-yl)oxy)acrylic acid;
or a pharmaceutically acceptable salt thereof.

In some embodiments, the ASBT inhibitor is a compound selected from:

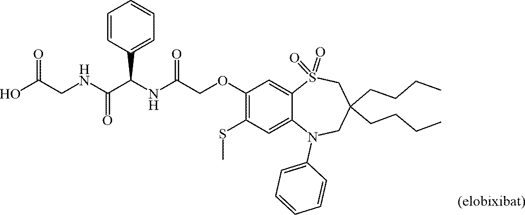

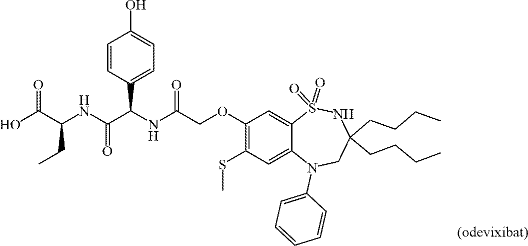

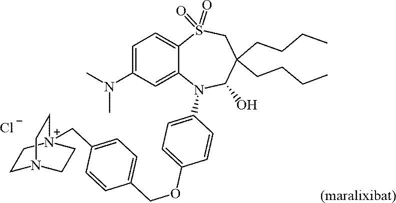

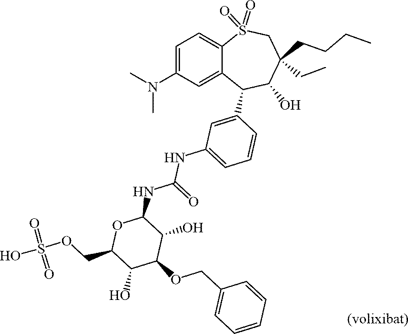

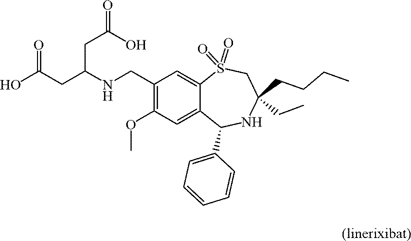

or a pharmaceutically acceptable salt thereof.

In some embodiments, the ASBT inhibitor is elobixibat, or a pharmaceutically acceptable salt thereof. In some embodiments, the ASBT inhibitor is odevixibat, or a pharmaceutically acceptable salt thereof. In some embodiments, the ASBT inhibitor is maralixibat, or a pharmaceutically acceptable salt thereof. In some embodiments, the ASBT inhibitor is volixibat, or a pharmaceutically acceptable salt thereof. In some embodiments, the ASBT inhibitor is linerixibat, or a pharmaceutically acceptable salt thereof. In some embodiments, the ASBT inhibitor comprises a combination of two or more of elobixibat, odevixibat, maralixibat, volixibat, and linerixibat, or a pharmaceutically acceptable salt thereof.

As used herein, the term “halo” refers to fluoro, chloro, bromo and iodo.

As used herein, the term “C₁₋₆alkyl” refers to a straight or branched alkyl group having from 1 to 6 carbon atoms, and the term “C₁₋₄alkyl” refers to a straight or branched alkyl group having from 1 to 4 carbon atoms. Examples of C₁₋₄alkyl include methyl, ethyl, n-propyl, isopropyl, n-butyl, isobutyl, sec-butyl and tert-butyl.

As used herein, the term “C₁₋₄haloalkyl” refers to a straight or branched C₁₋₄alkyl group, as defined herein, wherein one or more hydrogen atoms have been replaced with halogen. Examples of C₁₋₄haloalkyl include chloromethyl, fluoroethyl and trifluoromethyl.

As used herein, the terms “C₁₋₄alkoxy” and “C₁₋₄alkylthio” refer to a straight or branched C₁₋₄alkyl group attached to the remainder of the molecule through an oxygen or sulphur atom, respectively.

As used herein, the term “C₃₋₆cycloalkyl” refers to a monocyclic saturated hydrocarbon ring having from 3 to 6 carbon atoms. Examples of C₃₋₆cycloalkyl include cyclopropyl, cyclobutyl, cyclopentyl and cyclohexyl.

The term “amino” refers to an -NH₂ group. As used herein, the terms “N-(C₁₋₄alkyl)amino” and “N,N-di(C₁₋₄alkyl)amino” refer to an amino group wherein one or both hydrogen atom(s), respectively, are replaced with a straight or branched C₁₋₄alkyl group. Examples of N-(C₁₋₄alkyl)amino include methylamino, ethylamino and tert-butylamino, and examples of N,N-di-(C₁₋₄alkyl)amino include dimethylamino and diethylamino.

The term “aryl” denotes an aromatic monocyclic ring composed of 6 carbon atoms or an aromatic bicyclic ring system composed of 10 carbon atoms. Examples of aryl include phenyl, naphthyl and azulenyl.

As used herein, the term “N-(aryl-C₁₋₄alkyl)amino” refers to an amino group wherein a hydrogen atom is replaced with an aryl-C₁₋₄alkyl group. Examples of N-(aryl-C₁₋₄alkyl)amino include benzylamino and phenylethylamino. The term “C₁₋₆alkylcarbonylamino” refers to an amino group wherein a hydrogen atom is replaced with a C₁₋₆alkylcarbonyl group. Examples of C₁₋₆alkanoylamino include acetylamino and tert-butylcarbonylamino. The term “C₁₋₄alkyloxycarbonylamino” refers to an amino group wherein a hydrogen atom is replaced with a C₁₋₄alkyloxycarbonyl group. An example of C₁₋₄ alkyloxycarbonylamino is tert-butoxycarbonylamino. The terms “C₁₋₄alkylsulfonamido” and “C₃₋₆cycloalkylsulfonamido” refer to an amino group wherein a hydrogen atom is replaced with a C₁₋₄alkylsulfonyl or a C₃₋₆ cycloalkylsulfonyl group, respectively.

Some ASBT inhibitors, or pharmaceutically acceptable salts thereof, may have chiral centres and/or geometric isomeric centres (E- and Z-isomers). It is to be understood that the invention encompasses all such optical isomers, diastereoisomers and geometric isomers that possess ASBT inhibitory activity. The invention also encompasses any and all tautomeric forms that possess ASBT inhibitory activity. Certain ASBT inhibitors, or pharmaceutically acceptable salts thereof, may exist in unsolvated as well as solvated forms, such as, for example, hydrated forms. It is to be understood that the invention encompasses all such solvated forms that possess ASBT inhibitory activity.

As used herein, the term “pharmaceutically acceptable” refers to those compounds, materials, compositions and/or dosage forms that are suitable for human pharmaceutical use and that are generally safe, non-toxic and neither biologically nor otherwise undesirable.

A suitable pharmaceutically acceptable salt of an ASBT inhibitor is, for example, a base-addition salt of such a compound which is sufficiently acidic, such as an alkali metal salt (e.g., a sodium or potassium salt), an alkaline earth metal salt (e.g., a calcium or magnesium salt), an ammonium salt, or a salt with an organic base which affords a physiologically acceptable cation, for example a salt with methylamine, dimethylamine, trimethylamine, piperidine, morpholine or tris-(2-hydroxyethyl)amine.

### Liver and Renal Diseases

A liver disease as defined herein is any disease in the liver and in organs connected therewith, such as the pancreas, portal vein, the liver parenchyma, the intrahepatic biliary tree, the extrahepatic biliary tree, and the gall bladder. In some embodiments, the liver or renal disease or disorder is a bile acid-dependent disease or disorder, i.e., wherein bile acids are involved in the development or progression of the disease or disorder.

In some embodiments, the liver or renal disease or disorder comprises impaired or defective biliary flow.

In some embodiments, the liver or renal disease or disorder comprises cholestasis. In some embodiments, the accumulation of bile acids occurs in the extrahepatic biliary tree (extrahepatic or obstructive cholestasis). In some embodiments, the accumulation of bile acids occurs in the intrahepatic biliary tree (intrahepatic cholestasis).

In some embodiments, the liver disease or disorder is PFIC, type 2. PFIC-2 is caused by impaired bile salt secretion due to mutations in the ABCB11 gene, which codes for a protein known as BSEP (bile salt export pump), that moves bile acids out of the liver. Subjects with PFIC-2 often develop liver failure within the first few years of life and are at increased risk of developing a type of liver cancer known as hepatocellular carcinoma.

Van Wessel et al. (J. Hepatol. 2020, vol. 73, p. 84-93) have categorized BSEP deficiency into three groups, based on the type of mutation of the ABCB11 gene. BSEP1 patients have at least one p.D482G (c.1445A>G) or p.E297G (c.890A>G) mutation. With either of these mutations, some BSEP protein is still produced yet not enough. BSEP2 patients have at least one missense mutation, but not p.D482G or p.E297G. Many different mutations have been found to exist in this group of patients. Finally, BSEP3 patients have mutations that are known or predicted to lead to a non-functioning BSEP protein or to absent BSEP expression. The type of mutation of the ABCB11 gene is related to the severity of the disease, with BSEP1 deficiency causing less severe disease and BSEP3 deficiency causing the most severe disease. In some embodiments of the invention, the liver disease or disorder comprises a BSEP3 deficiency.

In some embodiments, the liver disease or disorder comprises biliary obstruction, which is a blockage of one or more bile ducts. Biliary obstruction may be caused by inflammation of the bile ducts (leading to cholangitis); by gallstones (leading to choledocholithiasis); or by tumours and neoplasms of the liver (e.g., liver cancer), of the biliary tract (such as cholangiocarcinoma and gallbladder cancer) or of the pancreas (pancreatic cancer). Alternatively, the biliary obstruction is caused by a congenital or condition, such as biliary atresia. Biliary atresia is a rare pediatric liver disease involving a partial or total blockage of large bile ducts, or even a complete absence of large bile ducts. This blockage or absence causes cholestasis that leads to the accumulation of bile acids that damages the liver. In some embodiments, the accumulation of bile acids occurs in the extrahepatic biliary tree. In some embodiments, the accumulation of bile acids occurs in the intrahepatic biliary tree. There are currently no approved drug therapies for this disorder. The current standard of care is the Kasai procedure, which is a surgery that removes the blocked bile ducts and directly connects a portion of the small intestine to the liver. Regardless, problems with impaired bile flow and accumulation of bile acids in the liver typically remain, and most patients will eventually need a liver transplant. Treatment of biliary atresia therefore also includes treatment of post-Kasai biliary atresia and post-liver transplantation biliary atresia.

In some embodiments, the liver disease or disorder is due to inflammation of the bile ducts, such as in acute cholangitis and obstructive cholangitis. The inflammation is often the result of an obstruction of the biliary tract, such as gallstones, but may also be caused by e.g., a tumour or a blood infection, or occur after liver or gallbladder endoscopy.

In some embodiments, the liver disease or disorder is malignant biliary obstruction, such as due to cholangiocarcinoma, pancreatic cancer (e.g., pancreatic adenocarcinoma), gallbladder cancer or colon cancer.

ASBT inhibitors may play a crucial role in mediating the toxic effects of bile acids in the kidneys. It has been observed that ASBT is strongly downregulated after bile duct litigation (BDL) in mice, and that inhibition of renal ASBT drastically ameliorates cholemic nephropathy in mice. Inhibition of ASBT may therefore have a protective effect on the kidneys, not least in conditions where patients also suffer from advanced liver diseases.

In some embodiments, the renal disease or disorder is a bile acid-dependent disease or disorder, i.e., wherein bile acids are involved in the development or progression of the disease or disorder.

In some embodiments, the renal disease or disorder is selected from the group consisting of cholemic nephropathy, chronic nephropathy, hyperbilirubinemia, renal dysfunction of obstructive jaundice, aging-induced impaired mitochondrial functions in the kidney, renal inflammation, acute kidney injury (AKI), kidney ischemia/reperfusion injury (IRI), chronic kidney disease (CKD), polycystic kidney disease (PKD), arthrogryposis-renal dysfunction-cholestasis (ARC) syndrome, familial lecithin cholesterol acyltransferase (LCAT) deficiency, chronic renal insufficiency, end-stage renal disease (ESRD), proximal tubule damage in the kidney, hepatorenal syndrome type 1, hepatorenal syndrome type 2, and acute-on-chronic liver disease.

In some embodiments, the renal disease or disorder is cholemic nephropathy, which is a state of kidney injury/failure in patients with obstructive jaundice. Also referred to as bile cast nephropathy, bile acid nephropathy, icteric nephrosis/nephropathy or jaundice-related nephropathy, cholemic nephropathy represents an underestimated but important cause of renal dysfunction in cholestasis or advanced liver diseases with jaundice. It is a common complication in patients with liver diseases such as liver cirrhosis, alcoholic steatohepatitis, drug-induced cholestatic liver injury and fulminant hepatitis, and is associated with high morbidity and mortality. Cholemic nephropathy is characterized by hemodynamic changes in the liver, kidney, systemic circulation, intratubular cast formation, and tubular epithelial cell injury, but the underlying pathophysiological mechanisms are still insufficiently understood.

Toxic bile acids have been suggested to play a role in the development of kidney injury in cholestasis (Fickert et al., Hepatology 2013, vol. 58, p. 2056-2069; Krones et al., Dig. Dis. 2015, vol. 33, p. 367-375; Tinti et al., Life 2021, vol. 11, 1200). The less toxic bile acid nor-ursodeoxycholic acid was shown to ameliorate kidney injury, and has been suggested as medical treatment for cholemic nephropathy (Krones et al., J Hepatol. 2017, vol. 67, p. 110-119). There currently is no specific treatment available for this condition.

In some embodiments, the subcutaneous administration of an ASBT inhibitor is combined with the oral administration of an ASBT inhibitor, such as a non-systemically available ASBT inhibitor, or with the oral administration of an LBAT inhibitor. Such combined treatment may have an additive or synergistic effect, and may result in the excretion of even larger amounts of bile acids. Examples of non-systemically available ASBT inhibitors include, but are not limited to, elobixibat, odevixibat, maralixibat, volixibat and linerixibat. The systemic absorption following oral administration of these ASBT inhibitors is less than 10%. Further examples of suitable ASBT inhibitors are disclosed in e.g., WO 2019/234077, WO 2020/161216, WO 2020/161217, WO 2021/110884, WO 2021/110885, WO 2021/110886, WO 2021/110887 and WO 2022/029101. Examples of suitable LBAT inhibitors are disclosed in e.g., WO 2021/110883, WO 2022/117778 and WO 2022/253997.

In some embodiments, the patient does not respond to treatment with an orally administered, non-systemically available ASBT inhibitor. As the subcutaneous administration of an ASBT inhibitor leads to modulation of the renal ASBT, it is believed that subcutaneous administration of an ASBT inhibitor may result in a stronger ASBT modulating effect than oral administration of said compound.

In some embodiments, the patient does not tolerate treatment with an orally administered, non-systemically available ASBT inhibitor, for instance when the patient experiences severe side effects such as severe diarrhoea. Because subcutaneous administration of an ASBT inhibitor also results in modulation of the renal ASBT, bile acids are excreted not only in stools but also in urine. This is expected to lead to a reduction in the incidence of diarrhoea.

Also provided herein is a method for treating a liver or renal disease or disorder in a subject in need thereof, the method comprising subcutaneously administering to the subject a therapeutically effective amount of an ASBT inhibitor. Also provided herein is the use of an ASBT inhibitor in the manufacture of a medicament for the treatment of a liver or renal disease or disorder, wherein the ASBT inhibitor is administered subcutaneously.

In some embodiments, following subcutaneous administration of the ASBT inhibitor, or a pharmaceutically acceptable salt thereof, the subject exhibits a reduction in serum bile acid concentration of at least 50% relative to baseline (e.g., at least 55%; at least 60; at least 65%; at least 70%; at least 75%; at least 80%; at least 85%; at least 90%; or at least 95%). In some embodiments, the subject exhibits a reduction in serum bile acid concentration of at least 60%, at least 70%, at least 80%, or at least 90% relative to baseline. In some embodiments, following subcutaneous administration of the ASBT inhibitor, or a pharmaceutically acceptable salt thereof, the subject exhibits a reduction in serum bile acid concentration of about 50% to about 95% relative to baseline (e.g., about 50% to about 90%, about 50% to about 85%, about 50% to about 80%, about 50% to about 75%, about 50% to about 70%, about 50% to about 65%, about 50% to about 60%, about 50% to about 55%, about 55% to about 95%, about 55% to about 90%, about 55% to about 85%, about 55% to about 80%, about 55% to about 75%, about 55% to about 70%, about 55% to about 65%, about 55% to about 60%, about 60% to about 95%, about 60% to about 90%, about 60% to about 85%, about 60% to about 80%, about 60% to about 75%, about 60% to about 70%, about 60% to about 65%, about 65% to about 95%, about 65% to about 90%, about 65% to about 85%, about 65% to about 80%, about 65% to about 75%, about 65% to about 70%, about 70% to about 95%, about 70% to about 90%, about 70% to about 85%, about 70% to about 80%, about 70% to about 75%, about 75% to about 95%, about 75% to about 90%, about 75% to about 85%, about 75% to about 80%, about 80% to about 95%, about 80% to about 90%, about 80% to about 85%, about 85% to about 95%, about 85% to about 90%, or about 90% to about 95% relative to baseline). In some embodiments, the serum bile acid concentration is normalized following subcutaneous administration of the ASBT inhibitor, or a pharmaceutically acceptable salt thereof. In some embodiments, the serum bile acid concentration is normalized following subcutaneous administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc.

In some embodiments, following subcutaneous administration of the ASBT inhibitor, or a pharmaceutically acceptable salt thereof, the subject exhibits an increase in urinary bile acids of at least 50% relative to baseline (e.g., at least 55%; at least 60; at least 65%; at least 70%; at least 75%; at least 80%; at least 85%; at least 90%; or at least 95%). In some embodiments, the subject exhibits an increase in urinary bile acids of at least 60%, at least 70%, at least 80%, or at least 90% relative to baseline. In some embodiments, following subcutaneous administration of the ASBT inhibitor, or a pharmaceutically acceptable salt thereof, the subject exhibits an increase in urinary bile acids of about 50% to about 95% relative to baseline (e.g., about 50% to about 90%, about 50% to about 85%, about 50% to about 80%, about 50% to about 75%, about 50% to about 70%, about 50% to about 65%, about 50% to about 60%, about 50% to about 55%, about 55% to about 95%, about 55% to about 90%, about 55% to about 85%, about 55% to about 80%, about 55% to about 75%, about 55% to about 70%, about 55% to about 65%, about 55% to about 60%, about 60% to about 95%, about 60% to about 90%, about 60% to about 85%, about 60% to about 80%, about 60% to about 75%, about 60% to about 70%, about 60% to about 65%, about 65% to about 95%, about 65% to about 90%, about 65% to about 85%, about 65% to about 80%, about 65% to about 75%, about 65% to about 70%, about 70% to about 95%, about 70% to about 90%, about 70% to about 85%, about 70% to about 80%, about 70% to about 75%, about 75% to about 95%, about 75% to about 90%, about 75% to about 85%, about 75% to about 80%, about 80% to about 95%, about 80% to about 90%, about 80% to about 85%, about 85% to about 95%, about 85% to about 90%, or about 90% to about 95% relative to baseline).

In some embodiments, the presence of a disease recited herein, such as biliary atresia or cholemic nephropathy, is determined by one or more biomarkers indicative of one or more of bile duct obstruction, cholestasis, inflammation, liver fibrosis, liver cirrhosis and/or scoring systems thereof. In some embodiments, the severity of a disease recited herein, such as biliary atresia or cholemic nephropathy, is determined by one or more biomarkers indicative of one or more of bile duct obstruction, cholestasis, inflammation, liver fibrosis, liver cirrhosis and/or scoring systems thereof. In some embodiments, the result of the treatment of a disease recited herein, such as biliary atresia or cholemic nephropathy, is determined by one or more biomarkers indicative of one or more of bile duct obstruction, cholestasis, inflammation, liver fibrosis, liver cirrhosis and/or scoring systems thereof. Non-limiting examples of biomarkers indicative of one or more of bile duct obstruction, cholestasis, inflammation, liver fibrosis, liver cirrhosis and/or scoring systems thereof include levels of alanine transaminase (ALT), aspartate transaminase (AST), alkaline phosphatase (ALP), gamma-glutamyl transferase (GGT), serum bilirubin, prothrombin time (PT), the international normalized ratio (INR), total protein and albumin (see, e.g., Lala et al., “Liver Function Tests.” StatPearls, StatPearls Publishing, 5 Oct. 2022 (PMID: 29494096), which is incorporated by reference herein in its entirety). In some embodiments, the subject exhibits an improvement in liver parameters (biomarkers) following administration of the ASBT inhibitor, or a pharmaceutically acceptable salt thereof. In some embodiments, the level of alkaline phosphatase (ALP) does not increase. In some embodiments, the level of alkaline phosphatase (ALP) decreases. In some embodiments, the “level” of an enzyme refers to the concentration of the enzyme, e.g., within blood. For example, the level of ALP can be expressed as Units/L.

In some embodiments, serum total bilirubin levels are decreased following subcutaneous administration of the ASBT inhibitor, or a pharmaceutically acceptable salt thereof. In some embodiments, total bilirubin levels are decreased about 0.5 mg/dL to about 15.0 mg/dL orabout 1 mg/dL to about 10.0 mg/dL ( e.g., about 2.0 mg/dL to about 10.0 mg/dL, about 3.0 mg/dL to about 10.0 mg/dL, about 4.0 mg/dL to about 10.0 mg/dL, about 5.0 mg/dL to about 10.0 mg/dL, about 6.0 mg/dL to about 10.0 mg/dL, about 7.0 mg/dL to about 10.0 mg/dL, about 8.0 mg/dL to about 10.0 mg/dL, about 9.0 mg/dL to about 10.0 mg/dL, about 1.0 mg/dL to about 9.0 mg/dL, about 2.0 mg/dL to about 9.0 mg/dL, about 3.0 mg/dL to about 9.0 mg/dL, about 4.0 mg/dL to about 9.0 mg/dL, about 5.0 mg/dL to about 9.0 mg/dL, about 6.0 mg/dL to about 9.0 mg/dL, about 7.0 mg/dL to about 9.0 mg/dL, about 8.0 mg/dL to about 9.0 mg/dL, about 1.0 mg/dL to about 8.0 mg/dL, about 2.0 mg/dL to about 8.0 mg/dL, about 3.0 mg/dL to about 8.0 mg/dL, about 4.0 mg/dL to about 8.0 mg/dL, about 5.0 mg/dL to about 8.0 mg/dL, about 6.0 mg/dL to about 8.0 mg/dL, about 7.0 mg/dL to about 8.0 mg/dL, about 1.0 mg/dL to about 7.0 mg/dL, about 2.0 mg/dL to about 7.0 mg/dL, about 3.0 mg/dL to about 7.0 mg/dL, about 4.0 mg/dL to about 7.0 mg/dL, about 5.0 mg/dL to about 7.0 mg/dL, about 6.0 mg/dL to about 7.0 mg/dL, about 1.0 mg/dL to about 6.0 mg/dL, about 2.0 mg/dL to about 6.0 mg/dL, about 3.0 mg/dL to about 6.0 mg/dL, about 4.0 mg/dL to about 6.0 mg/dL, about 5.0 mg/dL to about 6.0 mg/dL, about 1.0 mg/dL to about 5.0 mg/dL, about 2.0 mg/dL to about 5.0 mg/dL, about 3.0 mg/dL to about 5.0 mg/dL, about 4.0 mg/dL to about 5.0 mg/dL, about 1.0 mg/dL to about 4.0 mg/dL, about 2.0 mg/dL to about 4.0 mg/dL, about 3.0 mg/dL to about 4.0 mg/dL, about 1.0 mg/dL to about 3.0 mg/dL, about 2.0 mg/dL to about 3.0 mg/dL, or about 1.0 mg/dL to about 2.0 mg/dL) from baseline following subcutaneous administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, total bilirubin can be reduced at least 70% (e.g., approximately 99%) following subcutaneous administration of the ASBT inhibitor, or a pharmaceutically acceptable salt thereof, for at least 24 weeks. In some embodiments, total bilirubin levels are decreased about 3.0 mg/dL, about 4.0 mg/dL, about 5.0 mg/dL, about 6.0 mg/dL, about 7.0 mg/dL, about 8.0 mg/dL, or about 9.0 mg/dL from baseline following subcutaneous administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, total bilirubin can be reduced at least 70% (e.g., approximately 99%) following subcutaneous administration of the ASBT inhibitor, or a pharmaceutically acceptable salt thereof, for at least 24 weeks.

In some embodiments, serum alkaline phosphatase (ALP) levels are improved following subcutaneous administration of the ASBT inhibitor, or a pharmaceutically acceptable salt thereof. In some embodiments, ALP levels are decreased following subcutaneous administration of the ASBT inhibitor, or a pharmaceutically acceptable salt thereof. In some embodiments, ALP levels are decreased about 50 U/L to about 175 U/L, about 50 U/L to about 150 U/L, about 50 U/L to about 125 U/L, about 50 U/L to about 100 U/L, about 50 U/L to about 75 U/L, about 75 U/L to about 175 U/L, about 75 U/L to about 150 U/L, about 75 U/L to about 125 U/L, about 75 U/L to about 100 U/L, about 100 U/L to about 175 U/L, about 100 U/L to about 150 U/L, about 100 U/L to about 125 U/L, about 125 U/L to about 175 U/L, about 125 U/L to about 150 U/L, or about 150 U/L to about 175 U/L from baseline following subcutaneous administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, ALP levels can be reduced approximately 50%, approximately 60% or approximately 70% following subcutaneous administration of the ASBT inhibitor, or a pharmaceutically acceptable salt thereof, for at least 24 weeks.

In some embodiments, following subcutaneous administration of the ASBT inhibitor, or a pharmaceutically acceptable salt thereof, the subject exhibits a reduction in relative mRNA expression of kidney injury molecule-1 (KIM-1). In some embodiments, the subject exhibits a reduction in relative mRNA expression of urinary KIM-1 of between about 5% and about 100%, such as between about 10% and about 100%, or such as about 15% and about 100%. In some embodiments, the subject exhibits a reduction in relative mRNA expression of urinary KIM-1 of at least 50% (e.g., at least 55%; at least 60; at least 65%; at least 70%; at least 75%; at least 80%; at least 85%; at least 90%; or at least 95%). In some embodiments, the subject exhibits a reduction in relative mRNA expression of urinary KIM-1 of at least 60%, at least 70%, at least 80%, or at least 90%. In some embodiments, following subcutaneous administration of the ASBT inhibitor, or a pharmaceutically acceptable salt thereof, the subject exhibits a reduction in relative mRNA expression of urinary KIM-1 of about 50% to about 95% (e.g., about 50% to about 90%, about 50% to about 85%, about 50% to about 80%, about 50% to about 75%, about 50% to about 70%, about 50% to about 65%, about 50% to about 60%, about 50% to about 55%, about 55% to about 95%, about 55% to about 90%, about 55% to about 85%, about 55% to about 80%, about 55% to about 75%, about 55% to about 70%, about 55% to about 65%, about 55% to about 60%, about 60% to about 95%, about 60% to about 90%, about 60% to about 85%, about 60% to about 80%, about 60% to about 75%, about 60% to about 70%, about 60% to about 65%, about 65% to about 95%, about 65% to about 90%, about 65% to about 85%, about 65% to about 80%, about 65% to about 75%, about 65% to about 70%, about 70% to about 95%, about 70% to about 90%, about 70% to about 85%, about 70% to about 80%, about 70% to about 75%, about 75% to about 95%, about 75% to about 90%, about 75% to about 85%, about 75% to about 80%, about 80% to about 95%, about 80% to about 90%, about 80% to about 85%, about 85% to about 95%, about 85% to about 90%, or about 90% to about 95%).

In some embodiments, following subcutaneous administration of the ASBT inhibitor, or a pharmaceutically acceptable salt thereof, the subject exhibits a reduction in relative mRNA expression of lipocalin-2 (LCN2; also known as urinary neutrophil gelatinase-associated lipocalin, or NGAL). In some embodiments, the subject exhibits a reduction in relative mRNA expression of LCN2 between about 5% and about 100%, such as between about 10% and about 100%, or such as about 15% and about 100%. In some embodiments, following subcutaneous administration of the ASBT inhibitor, or a pharmaceutically acceptable salt thereof, the subject exhibits a reduction in relative mRNA expression of LCN2 of about 50% to about 100% (e.g., about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 100%, about 55% to about 95%, about 55% to about 90%, about 55% to about 85%, about 55% to about 80%, about 55% to about 75%, about 55% to about 70%, about 55% to about 65%, about 55% to about 60%, about 60% to about 100%, about 60% to about 95%, about 60% to about 90%, about 60% to about 85%, about 60% to about 80%, about 60% to about 75%, about 60% to about 70%, about 60% to about 65%, about 65% to about 95%, about 65% to about 100%, about 65% to about 95%, about 65% to about 90%, about 65% to about 85%, about 65% to about 80%, about 65% to about 75%, about 65% to about 70%, about 70% to about 100%, about 70% to about 95%, about 70% to about 90%, about 70% to about 85%, about 70% to about 80%, about 70% to about 75%, about 75% to about 100%, about 75% to about 95%, about 75% to about 90%, about 75% to about 85%, about 80% to about 100%, about 80% to about 95%, about 80% to about 90%, about 80% to about 85%, about 85% to about 95%, about 85% to about 90%, about 90% to about 100%, about 90% to about 95%, or about 95% to about 100%). In some embodiments, the subject exhibits a reduction in relative mRNA expression of LCN2 of at least about 50% (e.g., at least about 55%; at least about 60; at least about 65%; at least about 70%; at least about 75%; at least about 80%; at least about 85%; at least about 90%; or at least about 95%). In some embodiments, the subject exhibits a reduction in relative mRNA expression of LCN2 of at least about 60%, at least about 70%, at least about 80%, or at least about 90%.

### Formulation

The subcutaneous administration of the ASBT inhibitor according to the present invention requires a liquid aqueous formulation. Such formulations may comprise, in addition to the ASBT inhibitor, solubilizing and stabilizing excipients such as salts (e.g., saline), buffers, surfactants, cosolvents, antioxidants and preservatives.

Buffers may include salts such as phosphate, citrate, acetate, gluconate, lactate, tartrate, aspartate, glutamate and phthalate, or the corresponding acid forms thereof, as well as histidine or Tris (tris(hydroxymethyl)aminomethane). The pH of the liquid formulation is within the range of about 4 to about 9, more preferably within the range of about 5 to about 8, and even more preferably within the range of about 6 to 7.

The surfactant may be a cationic surfactant, an anionic surfactant or a nonionic surfactant. Examples of cationic surfactants include, but are not limited to, cetyltrimethylammonium bromide (cetrimonium bromide) and cetylpyridinium chloride. Examples of anionic surfactants include, but are not limited to, sodium dodecyl sulfate (sodium lauryl sulfate) and ammonium dodecyl sulfate (ammonium lauryl sulfate). Examples of nonionic surfactants include, but are not limited to, glycerol monooleate, glycerol monostearate, polyoxyl castor oil (Cremophor EL), poloxamers (e.g., poloxamer 407 or 188), polysorbate 80 and sorbitan esters (Tween). In a preferred embodiment, the surfactant is a cationic surfactant.

Examples of suitable cosolvents include, but are not limited to, ethanol, propylene glycol, polyethylene glycol 400 (PEG 400), N-methylpyrrolidone (NMP), dimethyl sulfoxide (DMSO), and N,N-dimethylacetamide (DMA).

Examples of suitable antioxidants include, but are not limited to, butylhydroxytoluene (BHT), ascorbyl palmitate, propyl gallate and ascorbic acid, and combinations thereof.

Examples of suitable preservatives include, but are not limited to, phenol, benzyl alcohol, methyl paraben, ethyl paraben, propyl paraben, ethylenediaminetetraacetic acid (EDTA), potassium sorbate and sodium benzoate, and combinations thereof.

In some embodiments, the concentration of the ASBT inhibitor in the liquid formulation is from about 0.001 to about 30 mg/mL. In some embodiments, the concentration of the ASBT inhibitor is from about 0.01 to about 10 mg/mL, such as from about 0.01 to about 1 mg/mL, about 0.01 to about 2 mg/mL, about 0.01 to about 3 mg/mL, about 0.01 to about 4 mg/mL, about 0.01 to about 5 mg/mL, about 0.01 to about 6 mg/mL, about 0.01 to about 7 mg/mL, about 0.01 to about 8 mg/mL, about 0.01 to about 9 mg/mL, about 1 to about 2 mg/mL, about 1 to about 3 mg/mL, about 1 to about 4 mg/mL, about 1 to about 5 mg/mL, about 1 to about 6 mg/mL, about 1 to about 7 mg/mL, about 1 to about 8 mg/mL, about 1 to about 9 mg/mL, about 1 to about 10 mg/mL, about 2 to about 3 mg/mL, about 2 to about 4 mg/mL, about 2 to about 5 mg/mL, about 2 to about 6 mg/mL, about 2 to about 7 mg/mL, about 2 to about 8 mg/mL, about 2 to about 9 mg/mL, about 2 to about 10 mg/mL, about 3 to about 4 mg/mL, about 3 to about 5 mg/mL, about 3 to about 6 mg/mL, about 3 to about 7 mg/mL, about 3 to about 8 mg/mL, about 3 to about 9 mg/mL, about 3 to about 10 mg/mL, about 4 to about 5 mg/mL, about 4 to about 6 mg/mL, about 4 to about 7 mg/mL, about 4 to about 8 mg/mL, about 4 to about 9 mg/mL, about 4 to about 10 mg/mL, about 5 to about 6 mg/mL, about 5 to about 7 mg/mL, about 5 to about 8 mg/mL, about 5 to about 9 mg/mL, about 5 to about 10 mg/mL, about 6 to about 7 mg/mL, about 6 to about 8 mg/mL, about 6 to about 9 mg/mL, about 6 to about 10 mg/mL, about 7 to about 8 mg/mL, about 7 to about 9 mg/mL, about 7 to about 10 mg/mL, about 8 to about 9 mg/mL, about 8 to about 10 mg/mL, or about 9 to about 10 mg/mL. In some embodiments, the concentration of the ASBT inhibitor in the liquid formulation is about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.2 mg/mL, about 1.4 mg/mL, about 1.6 mg/mL, about 1.8 mg/mL or about 2.0 mg/mL.

### Dose and Frequency of Administration

The ASBT inhibitor will normally be administered to a warm-blooded animal, such as a human, at a dose ranging from about 0.1 to about 1000 µg/kg/day, such as from about 1 µg/kg/day to about 1000 µg/kg/day, or such as from about 10 µg/kg/day to about 1000 µg/kg/day. In some embodiments, the ASBT inhibitor is administered at a dose of about 20 µg/kg/day, about 40 µg/kg/day, about 60 µg/kg/day, about 80 µg/kg/day, about 100 µg/kg/day, about 120 µg/kg/day, about 140 µg/kg/day, about 160 µg/kg/day, about 180 µg/kg/day, about 200 µg/kg/day, about 300 µg/kg/day, about 400 µg/kg/day, about 500 µg/kg/day, about 600 µg/kg/day, about 700 µg/kg/day, about 800 µg/kg/day, about 900 µg/kg/day, or about 1000 µg/kg/day.

In some embodiments, the ASBT inhibitor is administered at a dose of about 20 to about 800 µg/kg/day. For example, about 20 to about 600 µg/kg/day, about 20 to about 400 µg/kg/day, about 20 to about 200 µg/kg/day, about 20 to about 180 µg/kg/day, about 20 to about 160 µg/kg/day, about 20 to about 140 µg/kg/day, about 20 to about 120 µg/kg/day, about 20 to about 100 µg/kg/day, about 20 to about 80 µg/kg/day, about 20 to about 60 µg/kg/day, about 20 to about 40 µg/kg/day, about 40 to about 800 µg/kg/day, about 40 to about 600 µg/kg/day, about 40 to about 400 µg/kg/day, about 40 to about 200 µg/kg/day, about 40 to about 180 µg/kg/day, about 40 to about 160 µg/kg/day, about 40 to about 140 µg/kg/day, about 40 to about 120 µg/kg/day, about 40 to about 100 µg/kg/day, about 40 to about 80 µg/kg/day, about 40 to about 60 µg/kg/day, about 60 to about 800 µg/kg/day, about 60 to about 600 µg/kg/day, about 60 to about 400 µg/kg/day, about 60 to about 200 µg/kg/day, about 60 to about 180 µg/kg/day, about 60 to about 160 µg/kg/day, about 60 to about 140 µg/kg/day, about 60 to about 120 µg/kg/day, about 60 to about 100 µg/kg/day, about 60 to about 80 µg/kg/day, about 80 to about 800 µg/kg/day, about 80 to about 600 µg/kg/day, about 80 to about 400 µg/kg/day, about 80 to about 200 µg/kg/day, about 80 to about 180 µg/kg/day, about 80 to about 160 µg/kg/day, about 80 to about 140 µg/kg/day, about 80 to about 120 µg/kg/day, about 80 to about 100 µg/kg/day, about 100 to about 800 µg/kg/day, about 100 to about 600 µg/kg/day, about 100 to about 400 µg/kg/day, about 100 to about 200 µg/kg/day, about 100 to about 180 µg/kg/day, about 100 to about 160 µg/kg/day, about 100 to about 140 µg/kg/day, about 100 to about 120 µg/kg/day, about 120 to about 800 µg/kg/day, about 120 to about 600 µg/kg/day, about 120 to about 400 µg/kg/day, about 120 to about 200 µg/kg/day, about 120 to about 180 µg/kg/day, about 120 to about 160 µg/kg/day, about 120 to about 140 µg/kg/day, about 140 to about 800 µg/kg/day, about 140 to about 600 µg/kg/day, about 140 to about 400 µg/kg/day, about 140 to about 200 µg/kg/day, about 140 to about 180 µg/kg/day, about 140 to about 160 µg/kg/day, about 160 to about 800 µg/kg/day, about 160 to about 600 µg/kg/day, about 160 to about 400 µg/kg/day, about 160 to about 200 µg/kg/day, about 160 to about 180 µg/kg/day, about 180 to about 800 µg/kg/day, about 180 to about 600 µg/kg/day, about 180 to about 400 µg/kg/day, about 180 to about 200 µg/kg/day, about 200 to about 800 µg/kg/day, about 200 to about 600 µg/kg/day, about 200 to about 400 µg/kg/day, about 400 to about 800 µg/kg/day, about 400 to about 600 µg/kg/day, or about 600 to about 800 µg/kg/day of odevixibat, or a pharmaceutically acceptable salt thereof.

In some embodiments, the ASBT inhibitor is administered as a unit dose ranging from about 1 µg to about 100 mg, such as from about 10 µg to about 10 mg, such as from about 100 µg to about 2000 µg, or such as from about 200 µg to about 1500 µg. In some embodiments, the ASBT inhibitor is administered as a unit dose ranging from about 10 µg to about 9 mg, about 10 µg to about 8 mg, about 10 µg to about 7 mg, about 10 µg to about 6 mg, about 10 µg to about 5 mg, about 10 µg to about 4 mg, about 10 µg to about 3 mg, about 10 µg to about 2 mg, about 10 µg to about 1 mg, about 10 µg to about 800 µg, about 10 µg to about 600 µg, about 10 µg to about 400 µg, about 10 µg to about 200 µg, about 10 µg to about 100 µg, about 10 µg to about 50 µg, about 50 µg to about 10 mg, about 50 µg to about 9 mg, about 50 µg to about 8 mg, about 50 µg to about 7 mg, about 50 µg to about 6 mg, about 50 µg to about 5 mg, about 50 µg to about 4 mg, about 50 µg to about 3 mg, about 50 µg to about 2 mg, about 50 µg to about 1 mg, about 50 µg to about 800 µg, about 50 µg to about 600 µg, about 50 µg to about 400 µg, about 50 µg to about 200 µg, about 50 µg to about 100 µg, about 100 µg to about 10 mg, about 100 µg to about 9 mg, about 100 µg to about 8 mg, about 100 µg to about 7 mg, about 100 µg to about 6 mg, about 100 µg to about 5 mg, about 100 µg to about 4 mg, about 100 µg to about 3 mg, about 100 µg to about 2 mg, about 100 µg to about 1 mg, about 100 µg to about 800 µg, about 100 µg to about 600 µg, about 100 µg to about 400 µg, about 100 µg to about 200 µg, about 200 µg to about 10 mg, about 200 µg to about 9 mg, about 200 µg to about 8 mg, about 200 µg to about 7 mg, about 200 µg to about 6 mg, about 200 µg to about 5 mg, about 200 µg to about 4 mg, about 200 µg to about 3 mg, about 200 µg to about 2 mg, about 200 µg to about 1 mg, about 200 µg to about 800 µg, about 200 µg to about 600 µg, about 200 µg to about 400 µg, about 200 µg to about 10 mg, about 200 µg to about 9 mg, about 400 µg to about 8 mg, about 400 µg to about 7 mg, about 400 µg to about 6 mg, about 400 µg to about 5 mg, about 400 µg to about 4 mg, about 400 µg to about 3 mg, about 400 µg to about 2 mg, about 400 µg to about 1 mg, about 400 µg to about 800 µg, about 400 µg to about 600 µg, about 600 µg to about 10 mg, about 600 µg to about 9 mg, about 600 µg to about 8 mg, about 600 µg to about 7 mg, about 600 µg to about 6 mg, about 600 µg to about 5 mg, about 600 µg to about 4 mg, about 600 µg to about 3 mg, about 600 µg to about 2 mg, about 600 µg to about 1 mg, about 600 µg to about 800 µg, about 800 µg to about 10 mg, about 800 µg to about 9 mg, about 800 µg to about 8 mg, about 800 µg to about 7 mg, about 800 µg to about 6 mg, about 800 µg to about 5 mg, about 800 µg to about 4 mg, about 800 µg to about 3 mg, about 800 µg to about 2 mg, about 800 µg to about 1 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 10 mg, about 5 mg to about 9 mg, about 5 mg to about 8 mg, about 5 mg to about 7 mg, about 5 mg to about 6 mg, about 6 mg to about 10 mg, about 6 mg to about 9 mg, about 6 mg to about 8 mg, about 6 mg to about 7 mg, about 7 mg to about 10 mg, about 7 mg to about 9 mg, about 7 mg to about 8 mg, about 8 mg to about 10 mg, about 8 mg to about 9 mg, or about 9 mg to about 10 mg, In some embodiments, the ASBT inhibitor is administered as a unit dose of about 100 µg, about 200 µg, about 300 µg, about 400 µg, about 500 µg, about 600 µg, about 700 µg, about 800 µg, about 900 µg, about 1000 µg, about 1100 µg, about 1200 µg, about 1300 µg, about 1400 µg, about 1500 µg, about 1600 µg, about 1700 µg, about 1800 µg, about 1900 µg, or about 2000 µg. The frequency of administration can vary from once or twice a week to once or more times a day, such as two or three times daily. In some embodiments, the ASBT inhibitor is administered once daily. The frequency of administration can furthermore remain constant or be variable during the duration of the treatment. Several factors can influence the frequency of administration and the effective amount of the formulation that should be used for a particular treatment, such as the severity of the condition being treated, the duration of the treatment, as well as the age, weight, sex, diet and general medical condition of the patient being treated.

As used herein, the terms “treatment”, “treat” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The invention will now be described by the following examples which do not limit the invention in any respect. All cited documents and references are incorporated by reference.

### EXAMPLES

### Example 1

### Plasma Concentration Following Subcutaneous Administration

Elobixibat was administered to male C57BL/6 mice (n=5) as a subcutaneous injection at a dose of 3 or 10 mg/kg or as an intravenous injection at a dose of 1 mg/kg (single injections), or as repeated subcutaneous injections at a dose of 1 mg/kg at day 1 and day 5. Elobixibat was formulated in 70% PEG400; 10% ethanol; and 20% water and given at a dosing volume of 5 mL/kg subcutaneously or 1 mL/kg intravenously. Blood samples were collected after 0.25, 0.5, 1, 2, 4, 6, 8 and 24 hours following subcutaneous administration and after 0.083, 0.25, 0.5, 1, 2, 4, 6, 8 and 24 hours following intravenous administration. Blood samples were taken from saphenous vein. 0.2% EDTA was used as the anticoagulant. The samples were analyzed by a discovery grade bioanalytical method developed for the estimation of elobixibat in plasma, using an LC-MS/MS system.

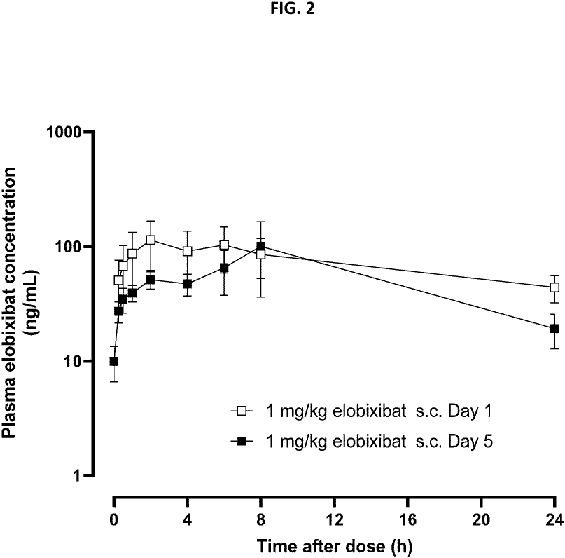

The results for single subcutaneous (3 or 10 mg/kg) and intravenous (1 mg/kg) administrations are shown in FIG. 1, and the results for repeated subcutaneous administrations (1 mg/kg at day 1 and day 5) are shown in FIG. 2.

### Example 2

### In Vivo Animal Model of Cholestatic Disease

Chronic feeding of 3,5-diethoxycarbonyl-1,4-dihydrocollidine (DDC) was used as an in vivo model for cholestatic liver injury. Chronic feeding of DDC produces an intraductal porphyrin plug which leads to ductular obstruction contributing to damage of biliary epithelia. The time course of the DDC model usually ranges from 1 to 6 weeks, depending on the severity of the cholangiopathy that the study requires. Chronic feeding of DDC in mice reproduces the main histopathological hallmarks of human cholestatic disease such as (1) remodeling of biliary compartments giving rise to ductular reaction, (2) periductular fibrosis, and (3) inflammatory infiltrate (Mariotti et al., Biochim Biophys Acta Mol Basis Dis 2018, vol. 1864, p. 1254-1261; Pose et al., Methods Mol. Biol. 2019, vol. 1981, p. 249-257).

Male C57BL/6J WT mice of 8 weeks old were used. After completion of quarantine and acclimatization period, animals were randomized based on body weight into experimental groups (n=7-9/group). Animals were fed chow (control diet) or chow plus 0.1% DDC (DDC diet) for 14 days, and treated once daily with vehicle (s.c.) or the ASBT inhibitor elobixibat at a dose of 0.3, 1.0 or 3.0 mg/kg (s.c.), as shown in Table 1. Animals were weighed at days 7, 11 and 14. Blood samples were collected at day 7. On day 11, animals were moved to individual cages with wire bottom for fecal collection. Blood, urine, tissue samples and 3-day fecal samples were collected at day 14.

TABLE 1

| Group No. | Diet | Drug | Dose | Route | Number of animals |
|---|---|---|---|---|---|
| 1 | control diet | vehicle | | s.c | 8 |
| 2 | control diet | elobixibat | 0.3 mg/kg | s.c | 7 |
| 3 | control diet | elobixibat | 1.0 mg/kg | s.c | 8 |
| 4 | control diet | elobixibat | 3.0 mg/kg | s.c | 9 |
| 5 | DDC diet | vehicle | | s.c | 8 |
| 6 | DDC diet | elobixibat | 0.3 mg/kg | s.c | 9 |
| 7 | DDC diet | elobixibat | 1.0 mg/kg | s.c | 9 |
| 8 | DDC diet | elobixibat | 3.0 mg/kg | s.c | 9 |

Endpoints measured at the end of the study include fecal bile acid excretion, urine and serum bile acid concentration and composition, and serum chemistries reflecting liver status.

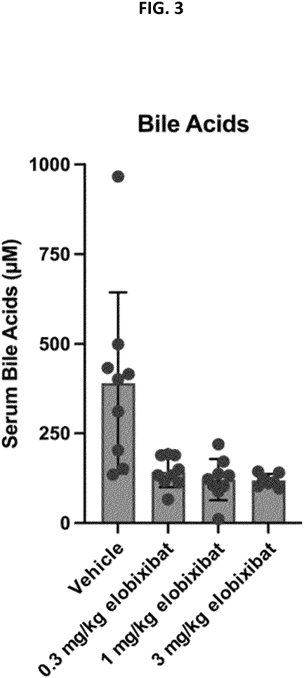

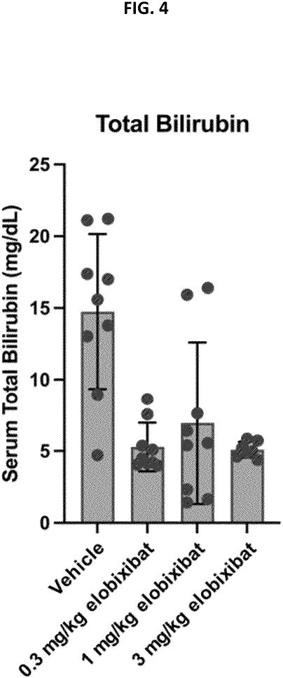

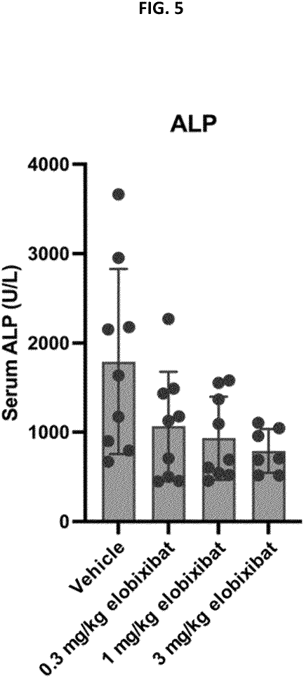

Blood sample analysis showed a statistically significant reduction in serum bile acids (FIG. 3) and serum total bilirubin (FIG. 4) with s.c. elobixibat daily administration. The levels are reduced at the lowest dose, and no additional reduction was observed at higher doses. A dose-dependent trend towards reduced serum alkaline phosphatase (ALP) levels was observed with s.c. elobixibat daily administration (FIG. 5).

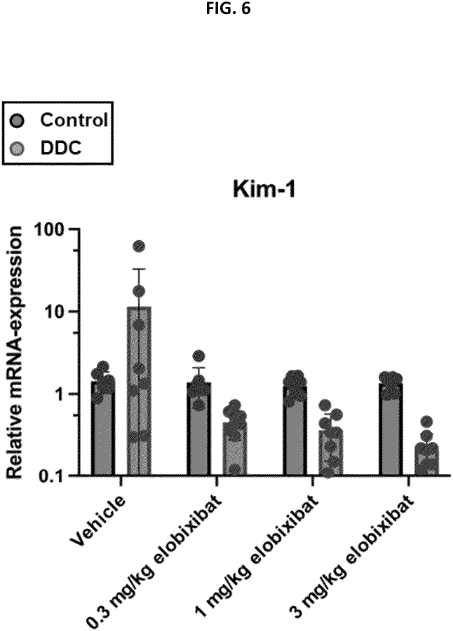

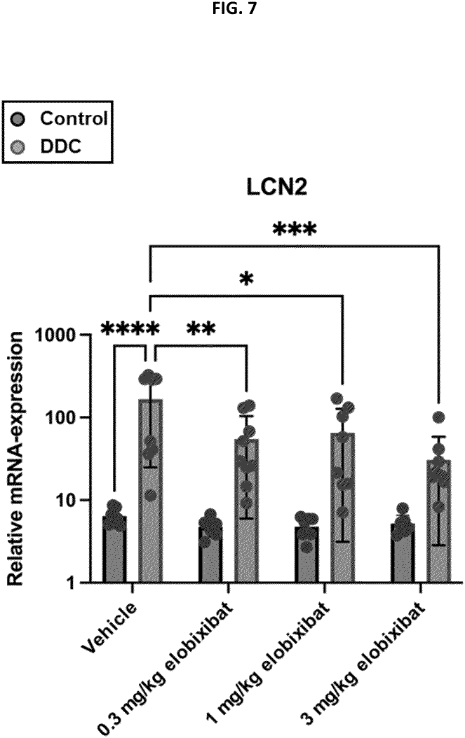

FIGS. 6 and 7 show the mRNA expression for markers of renal proximal tubule cell injury KIM-1 (kidney injury molecule-1) and LCN2 (lipocalin-2). mRNA expression for KIM-1 and LCN2 was induced in DDC cholestatic mice. It was observed that mRNA expression for KIM-1 and LCN2 was reduced in DDC cholestatic mice treated with s.c. elobixibat versus vehicle.

1-31. (canceled)

32. A method of treating a liver or renal disease or disorder in a subject, the method comprising subcutaneously administering to a subject in need thereof a therapeutically effective amount an ASBT inhibitor, or a pharmaceutically acceptable salt thereof.

33. The method according to claim 32, wherein the ASBT inhibitor does not inhibit renal ASBT at clinically relevant levels following oral administration of the ASBT inhibitor.

34. The method according to claim 32, wherein the ASBT inhibitor is less than 10% systemically absorbed following oral administration of the ASBT inhibitor.

35. The method according to claim 32, wherein the ASBT inhibitor is selected from the group consisting of elobixibat, odevixibat, maralixibat, volixibat and linerixibat, or a pharmaceutically acceptable salt thereof.

36. The method according to claim 32, wherein the ASBT inhibitor is elobixibat, or a pharmaceutically acceptable salt thereof.

37. The method according to claim 32, wherein the liver or renal disease or disorder is a bile acid-dependent disease or disorder.

38. The method according to claim 32, wherein the liver or renal disease or disorder comprises impaired or defective biliary flow.

39. The method according to claim 32, wherein the liver or renal disease or disorder comprises cholestasis.

40. The method according to claim 32, wherein the liver disease or disorder is PFIC, type 2.

41. The method according to claim 32, wherein the liver disease or disorder comprises a BSEP3 deficiency.

42. The method according to claim 32, wherein the liver or renal disease or disorder comprises biliary obstruction.

43. The method according to claim 32, wherein the liver disease or disorder is biliary atresia.

44. The method according to claim 43, wherein the biliary atresia comprises post-Kasai biliary atresia or post-liver transplantation biliary atresia.

45. The method according to claim 32, wherein the liver disease or disorder is acute cholangitis or obstructive cholangitis.

46. The method according to claim 32, wherein the liver disease or disorder is malignant biliary obstruction.

47. The method according to claim 46, wherein the malignant biliary obstruction is due to cholangiocarcinoma, pancreatic cancer, gallbladder cancer or colon cancer.

48. The method according to claim 32, wherein the renal disease or disorder is selected from the group consisting of cholemic nephropathy, chronic nephropathy, hyperbilirubinemia, renal dysfunction of obstructive jaundice, aging-induced impaired mitochondrial functions in the kidney, renal inflammation, acute kidney injury (AKI), kidney ischemia/reperfusion injury (IRI), chronic kidney disease (CKD), polycystic kidney disease (PKD), arthrogryposis-renal dysfunction-cholestasis (ARC) syndrome, familial lecithin cholesterol acyltransferase (LCAT) deficiency, chronic renal insufficiency, end-stage renal disease (ESRD), proximal tubule damage in the kidney, hepatorenal syndrome type 1, hepatorenal syndrome type 2, and acute-on-chronic liver disease.

49. The method according to claim 32, wherein the renal disease or disorder is cholemic nephropathy.

50. The method according to claim 32, wherein the method further comprises orally administering to the subject a non-systemically available ASBT inhibitor.

51. The method according to claim 32, wherein the subject does not respond to treatment with an orally administered, non-systemically available ASBT inhibitor.

52. The method according to claim 32, wherein the subject does not tolerate treatment with an orally administered, non-systemically available ASBT inhibitor.

53. The method according to claim 32, wherein the ASBT inhibitor is administered once daily.

54. The method according to claim 32, wherein the subject exhibits a reduction in serum bile acid concentration following subcutaneous administration of the IBAT inhibitor.

55. The method according to claim 54, wherein the reduction in serum bile acid concentration is at least 60% relative to baseline.

56. The method according to claim 32, wherein the subject exhibits an increase in urinary bile acids following subcutaneous administration of the IBAT inhibitor.

57. The method according to claim 56, wherein the increase in urinary bile acids is at least 60% relative to baseline.

58. The method according to claim 32, wherein the subject exhibits an improvement in one or more liver parameters following subcutaneous administration of the ASBT inhibitor.

59. The method according to claim 58, wherein the one or more liver parameters are selected from the group consisting of serum total bilirubin level, serum alkaline phosphatase (ALP) level, serum alanine aminotransferase (ALT) level and serum aspartate aminotransferase (AST) level.

60. The method according to claim 58, wherein the improvement in the one or more liver parameters occurs following subcutaneous administration of the ASBT inhibitor for at least 4 weeks.

61. The method according to claim 32, wherein the subject exhibits a reduction in relative mRNA expression of kidney injury molecule-1 (KIM-1) following subcutaneous administration of the ASBT inhibitor.

62. The method according to claim 61, wherein the reduction in relative mRNA expression of KIM-1 is at least 60%.

63. The method according to claim 32, wherein the subject exhibits a reduction in relative mRNA expression of lipocalin-2 (LCN2) following subcutaneous administration of the ASBT inhibitor.

64. The method according to claim 63, wherein the reduction in relative mRNA expression of LCN2 is at least 60%.
